(19) United States
(12) Patent Application Publication
(10) Pub. No.: US 2008/0299666 A1
(43) Pub. Date: Dec. 4, 2008

(54) Novel Peptide-Based Borono-Lectin (PBL) Sensors

(75) Inventors: John J. Lavigne, Columbia, SC (US); Paul R. Thompson, Columbia, SC (US)

Correspondence Address: DORITY & MANNING, P.A., POST OFFICE BOX 1449, GREENVILLE, SC, 29602-1449, US

(73) Assignee: University of South Carolina, Columbia, SC (US)

(21) Appl. No.: 12/130,334

(22) Filed: May 30, 2008

Related U.S. Application Data
(60) Provisional application No. 60/932,773, filed on Jun. 1, 2007.

(51) Int. Cl. G01N 33/68; G01N 33/50; C07K 2/00

(52) U.S. Cl. 436/87; 436/94; 530/300

(57) ABSTRACT

Peptide-based borono-lectin sensors, along with their synthesis and analysis and methods of use, are generally described. These sensors use peptides as a scaffold and introduce boronic acid moieties onto the peptide scaffold as the binding site for the targeted analyte (e.g., carbohydrates, glycans, etc.). The boronic acid moieties can be arranged on the protein scaffold in such a manner that a particularly targeted carbohydrate or glycoprotein will bond to the protein scaffold via the boronic acid functionality. Such bonding can indicated the presence or absence of that targeted analyte in the sample.

### PRIORITY INFORMATION

The present application claims priority to U.S. Provisional Application Serial No. 60/932,773 filed on May 30, 2007, entitled “Novel Peptide-based Borono-Lectin (PBL) Sensors”, the disclosure of which is incorporated by reference herein.

### BACKGROUND

It is of high significance to sense carbohydrates and glycoproteins because changes in glycosylation are closely associated with disease states such as cancer and inflammation. These changes in glycosylation include both the under- and overexpression of naturally-occurring glycans, as well as the neo-expression of glycans. It has been found that cancer cells frequently display glycans at different levels or with different structures from those observed on normal cells. There are two types of structural changes of glycans. One is an increase in the size or branching of N-linked glycans, which is a change in the core structure of the glycan. The second type of change is on terminal structures. Structural changes to glycans are a hallmark of the onset of cancer and inflammation.

A growing body of evidence supports the idea that during tumorgenesis aberrant glycosylation events occur to both cell surface and secreted glycoproteins and glycolipids and as a result the glycans produced by cancer cells differ in both structure and level to those produced by normal cells. These changes generally arise from the altered expression of glycosyl-transferases; typically leading to increased branching in core glycan structures as well as altering the terminal saccharide structures. The appearance of a variety of sialylated and fucosylated terminal glycan structures [e.g., Sialyl Lewis X (sLe^{x}), sialyl Lewis A (sLe^{a}), sialyl Tn (sTn), and Lewis Y (Le^{y})] has been associated with malignancy. While it is unclear whether these changes in glycan content are a cause or effect of oncogenesis, it is clear that specific cell surface glycans can contribute to the metastatic potential of particular tumor types. Regardless of their specific role in oncogenesis, the expression of these various glycan structures is dependent on both the tumor type and the stage of the disease; thus their appearance can be exploited for the development of novel cancer diagnostics.

Table 1.1 shows many of the changes observed in malignant tissues throughout the body. For example, the expression of polysialic acid (PSA) in colon cancer is negative; however, in breast cancer, this glycan is expressed. Malignant tissues, interestingly, have specific patterns of glycan expression. From Table 1.1, it is clear that breast cancer and colon cancer share the common overexpression of seven glycans (sLe^{x}, sLe^{a}, sTn, TF, Le^{y}, GloboH, and GM2) but differ in the expression of PSA. Another common feature of tumors is the overproduction of certain glycoproteins. For example, epithelial tumors often overproduce mucin glycoproteins.

TABLE 1.1
Common expression patterns of cancer glycans on malignant tissues

| Cancer | Malignant tissue | | | | | | | | |
|---|---|---|---|---|---|---|---|---|---|
| glycan | Ovary | Pancreas | Blood | Breast | Colon | Brain | Prostate | Skin | Lung |
| sLe^{x} | | x | | x | x | | | | x |
| sLe^{a} | | x | | x | x | | | | x |
| sTn | x | x | | x | x | | x | | x |
| TF | x | | | x | x | | x | | |
| Le^{y} | x | x | | x | x | | x | | x |
| GloboH | x | x | | x | x | | x | | x |
| PSA | | x | x | x | | x | | | x |
| GD2 | | | x | | | x | | x | |
| GD3 | | | | | | x | | x | |
| FucosylGM1 | | | | | | | | | x |
| GM2 | | | | | | | | | |

Undoubtedly, the development of sensors, which can detect glycans characteristic of disease conditions, will provide a powerful diagnostic tool of these diseases.

Significant effort has been made toward the development of sensors or sensing assays for carbohydrates and glycoproteins and these sensors or sensing assays can be generally classified into three categories including enzyme-based sensors, lectin-based sensors, and chemosensors. In an enzyme-based approach, carbohydrate detection generally relies on enzyme catalysis of saccharide substrates. The detected signal from enzyme-based sensors comes from the reaction products of the enzymic activity. Thus, the maintenance and optimization of biological activity of the enzyme are critical. Although enzyme assay methods have been well-established, they have several disadvantages. First, they tend to be complicated and expensive. Second, their stability is limited, which prevents them from long-term use. Lectin-based sensors operate through detection of binding activity between the targeted saccharides and lectins of the sensor. Some native lectin-based sensors lack good selectivity which prevents the accurate high-throughput quantization and assay of carbohydrates and glycoproteins. It has been reported that the introduction of an artificial sugar-binding site into a native lectin can modulate saccharide selectivity. In contrast, chemosensors generally include non-proteinaceous natural or synthesized detection compounds and can have higher stability compared with enzyme-based or lectin-based sensors. Among chemosensors, boronic acid-containing compounds have been widely investigated for many years.

The design of boronic acid-based sensors relies on the fact that boronic acids can bind tightly with 1,2 and 1,3-diols to form covalent yet reversible bonds generating five or six-member cyclic boronate esters, as illustrated in Scheme 1.1.

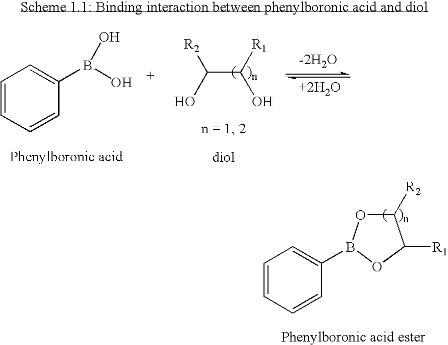

For a sensor, both a receptor and a signaling component are essential. The former interacts with target molecules to make the binding event happen and the latter can indicate or report the binding event. In boronic acid-based sugar sensors, the boronic acid groups will bind with sugars (diol-containing compounds) as the receptor. However, binding is not enough as there must be a detectable signal associated with the binding event to identify this binding. Fortunately, some detection methods have been developed for boronic acid-based sensors such as fluorescence, absorbance, CD (circular dichroism), and electrochemical measurement. Among them, the fluorescence detection methods are the most interesting because of their high sensitivity and enhanced response. Generally speaking, these fluorescence sensors have fallen into two major mechanistic categories: photoinduced electron transfer (PET) and intramolecular charge transfer (ICT).

Scheme 1.2 is an example of a PET fluorescence sensor. A boronic acid was used as the receptor and anthrancene as the reporter. The lone pair of electrons on nitrogen is known to quench the fluorescence of the anthrancene moiety. However, upon binding with diols to form boronate esters, the five membered ring is formed. The bond angles of this five membered ring are close to those of a tetrahedral structure. Thus, the geometry of the orbitals on boron approach that of sp3 hybridization and the Lewis acidity of the boron atom increases, which results in an increased B—N interaction. This interaction reduces the availability of the lone pairs of electrons on nitrogen for the photoinduced electron transfer process. Thus, the fluorescence intensity increases upon the diol binding. This is called an off-on PET system.

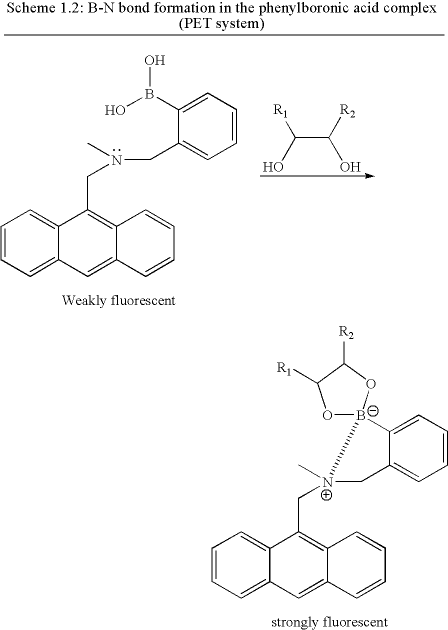

In order to improve the response of sugar sensors, it would be desirable if the binding could induce both the change of fluorescence intensity and shift of fluorescence emission. Unfortunately, most PET-based sensors provide only a change of fluorescence intensity. However, intramolecular charge transfer (ICT) systems have been found to be very sensitive to small perturbations such as binding with sugars, which can lead to changes in both the fluorescence intensity and a spectral shift. An example of an ICT system includes the compound 4′-Dimethylaminostilbene-4-boronic acid (DSTBA), which has the following structure:

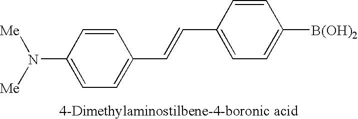

Upon binding with fructose at pH=8, the fluorescence intensity increases with the increase of the concentration of fructose and a 30 nm blue shift of the emission (480 nm to 450 nm) has also been observed. It is thought that the binding between the boronic acid and sugar forms anionic boron and thus the boronic acid group changes from an electron withdrawing group to an electron donating group affecting the ICT with the dimethylamine.

Various scaffolds have been used for boronic acid based chemosensors including anthrancene-based, porphyrin-based, and pyrene-based compounds. Moreover, the number and orientation of the boronic acid groups have also been found to be very important for binding selectivity. The selectivity of monoboronic acid hosts for monosaccharides is: fructose>galactose>glucose, i.e., the monoboronic acid compounds (compound A in FIG. 1.3) show better selectivity for fructose than either glucose or galactose.

A primary objective of early work in developing boronic acid-based chemosensors was to identify a better means to monitor the level of blood glucose for diabetes, i.e., sensors with better selectivities for glucose. However, it has been difficult to achieve with only a single boronic acid, but it has been possible for sensors with at least two boronic acids moieties. For instance, a compound (compound B in FIG. 1.3) has been developed that showed good selectivity for glucose. The observed selectivity has been attributed to the spatial distance of the two boronic acids being just right for glucose.

Recently, a series of diboronic acid compounds (FIG. 1.4.) were designed with varying rigidity and distance (different linkers) between the two boronic acids for selective recognition of sialyl Lewis X (sLe^{x}), a cancer-associated glycan. In the design, the key was to identify an appropriate linker with the proper distance and orientation of two boronic acids to fit the more complex carbohydrates. The result indicated that when the linker is a phenyl ring, the compound was able to fluorescently label cells expressing high levels of sLe^{x} within a concentration range of 0.5 to 10 μM. The unique spatial relationship of the two boronic acids allows for more favorable interactions with sLe^{x} compared to other diboronic acid compounds prepared. So, the geometry or orientation of diboronic acid compounds is crucial for selectivity. Furthermore, multiple boronic acid-based fluorescence sensors have been studied and show that multiple boronic acids can enhance the affinity of binding.

The design of most boronic acid-based chemosensors focuses on the synthesis of organic molecules or polymers containing boronic acid moieties. There are several important problems to be addressed prior to further applications. First, most designed boronic acid-based sensors are poorly water-soluble and thus biologically incompatible because they mainly contain hydrophobic polycyclic aromatic compounds. This poses a huge obstacle for their use as sensors because, for carbohydrate recognition, an aqueous testing environment is commonly used. Second, the synthesis of these polycyclic aromatic scaffolds is by no means trivial. Third, most sensors are based on large aromatic fluorophores which are toxic or even carcinogenic themselves. Therefore, a need exists for a biosensor that addresses these issues.

### SUMMARY

Objects and advantages of the invention will be set forth in part in the following description, or may be obvious from the description, or may be learned through practice of the invention.

In general, the present disclosure is directed toward novel peptide-based borono-lectin (“PBL”) sensors, along with their synthesis and analysis, are described.

Other features and aspects of the present invention are discussed in greater detail below.

### Definitions

Carbohydrates are a general term that refers to monosaccharides, oligosaccharides, and polysaccharides. Monosaccharides are polyhydroxy aldehydes or ketones composed of 3-9 carbon atoms including one or multiple stereochemical carbon centers. A few examples of monosaccharides are glucose, galactose, fructose, mannose, ribose, and deoxyribose. Covalently linked monosaccharides are commonly referred to as oligosaccharides (2-10 monosaccharide residues), polysaccharides (10-20 monosaccharide residues), and even bigger carbohydrates.

Almost all monosaccharides spontaneously cyclize to form relatively more energetically stable structures. Owing to the stability of five or six-membered rings, furanoses and pyranoses are the dominant structures corresponding to the heterocyclic compounds furan and pyran. The formation of the cyclic forms leads to two stereo stereochemically distinct structures: anomers α and β. Anomer α means that the hydroxyl group at the anomeric position is in the axial position; β means that this hydroxyl group is in the equatorial position. Meanwhile, the symbols D and L are assigned to define the absolute configuration of the asymmetric carbon farthest from the aldehyde or keto group. In Fisher projections, if the hydroxyl group is placed on the right-hand side of the asymmetric central carbon atom, the monosaccharide is termed the D-form; conversely, it is the L-form.

Carbohydrates play important roles in all forms of life as evidenced in the following aspects. For example, carbohydrates are metabolic intermediates and are a storage form of energy. Additionally, carbohydrates are important structural elements of RNA and DNA and cell walls of bacteria and plants. Carbohydrates are also principal players in mediating both interactions among cells and interactions between cells and other cellular elements. Hence, it is understandable that the structures, the placement of carbohydrates at specific sites within proteins and the functions of carbohydrates are critical in the life of all organisms. Carbohydrates are closely associated with human diseases.

Glycoproteins are proteins with covalently bound carbohydrates. The term Glycan is used in one embodiment to refer to the carbohydrate portion of a glycoconjugate, e.g., a glycoprotein, a glycolipid or a proteoglycan. The term can also refer to oligo- or polysaccharides. The proportion of glycans in glycoproteins varies widely from 2-3 wt % to as high as 90 wt % such as in some epithelial mucins. Glycans can affect the intrinsic properties of their linked proteins. It has been found that the abnormal expression of glycan levels or structures is often a hallmark of disease states. FIG. 1.1 depicts some exemplary cancer-associated glycans: eLe^{x}, sLe^{a}, Le^{x}, sLe^{y}, Tn, and sTn. Other cancer-associated glycans are known to those skilled in the art.

Glycosylation is used to describe the process or result of linking saccharides to another substrate, such as proteins. Based on the linking styles, there are two types of glycoproteins: N-linked glycoproteins and O-linked glycoproteins, which are illustrated in FIG. 1.2.

In N-linked glycoproteins, an oligosaccharide is linked to the protein through a N-acetylglucosamine (GlcNAc) molecule. The GlcNAc is attached through a β-N-glycosidic-type bond to the nitrogen of the amide group of the side chain of an asparagine (Asn) on the polypeptide chain. In O-linked glycoproteins, the oligosaccharide is linked through a GalNAc molecule in an α-O-glycosidic type bond to an oxygen of a serine or threonine on the polypeptide chain.

Natural lectins are naturally occurring proteins or glycoproteins of non-immune origin which bind carbohydrates non-covalently and reversibly with high specificity. They are found in most organisms such as viruses, bacteria, plants and animals. Other proteins that can interact with carbohydrates include enzymes and antibodies. Different from enzymes, lectins have no catalytic activity; in contrast to antibodies, lectins are not the products of an immune response. Lectins are often used as experimental tools to detect specific carbohydrates, especially glycoproteins. The main function of lectins is in cell recognition. They can recognize the change occurring on cell surfaces during physiological and pathological processes. These biological processes include: clearance of glycoproteins from the circulatory system; control of intracellular traffic of glycoproteins; adhesion of infectious agents to host cells; recruitment of leukocytes to inflammatory sites; and cell interactions in the immune system, in malignancy and metastasis. Therefore, lectins have been of great interest in biological research.

As used herein, the term “peptide-based borono-lectin” (“PBL”) refers to a synthetic material formed from amino acids with a boronic acid functionality that can be configured to bond to a specific diol material. As such, PBLs can be constructed to bind to specific carbohydrates or glycoproteins similar to natural lectins.

Natural amino acids are presented herein according to standard one or three letter symbols as follows:

amino acid symbols

| | One | Three |
|---|---|---|
| | letter | letter |
| Amino acid | symbol | symbol |
| alanine | A | Ala |
| arginine | R | Arg |
| asparagine | N | Asn |
| aspartic acid | D | Asp |
| cysteine | C | Cys |
| glutamic acid | E | Glu |
| glutamine | Q | Gln |
| glycine | G | Gly |
| histidine | H | His |
| isoleucine | I | Ile |
| leucine | L | Leu |
| lysine | K | Lys |
| methionine | M | Met |
| phenylalanine | F | Phe |
| proline | P | Pro |
| serine | S | Ser |
| threonine | T | Thr |
| tryptophan | W | Trp |
| tyrosine | Y | Tyr |
| valine | V | Val |

### BRIEF DESCRIPTION OF THE FIGURES

A full and enabling disclosure of the present invention, including the best mode thereof to one skilled in the art, is set forth more particularly in the remainder of the specification, which includes reference to the accompanying figures, in which:

FIG. 1.1 illustrates cancer-associated glycans;

FIG. 1.2 illustrates N-linked and O-linked glycoproteins;

FIG. 1.3 illustrates monoboronic acid and diboronic acid compounds as may be utilized in disclosed sensors;

FIG. 1.4 illustrates diboronic acid compounds as may be utilized in disclosed sensors;

FIG. 2.1 illustrates a flow chart of one embodiment of a design of peptide-based borono-lectin sensors as described herein;

FIG. 2.2 illustrates exemplary building blocks of a 7-mer peptide including BPA;

FIG. 2.3 illustrates exemplary building blocks of another 7-mer peptide as may be utilized as described herein;

FIG. 2.4 illustrates amino acids used in one embodiment of a library synthesis and schematic illustration of the procedure for synthesis of peptide library;

FIG. 2.5 illustrates the addition of a fluorescent dye onto protein to signal the binding to PBL

FIG. 2.6 is a 12% SDS-PAGE image of FITC-ovalbumin conjugate;

FIG. 2.7 illustrates the UV/vis absorption spectra of glycoproteins and their FITC;

FIG. 2.8 is a 12% SDS-PAGE image of FITC-ovalbumin conjugate;

FIG. 2.9 illustrates the UV/vis absorption spectrum of rhodamine-PSM;

FIG. 2.10 is a schematic representation of the on-bead screening of binding of PBLs as described herein;

FIG. 2.11 illustrates structures of Resorufin β-D-glucopyranoside and Resorufin β-D-Galactopyranoside;

FIG. 2.12 are images of screening PBLs with Resufin-β-D-glucopyranoside (upper) and Resufin-β-D-galacopyranoside (below);

FIG. 2.13 illustrates identification of non-specific binding (the concentration of CR-Ovalbumin=1000 μg/ml);

FIG. 2.14 illustrates non-specific binding and blocking;

FIG. 2.15 shows the detection limit of detection of FITC-ovalbumin;

FIG. 2.16 shows the detection limit of detection of FITC-BSM;

FIG. 2.17 shows the detection limit of detection of FITC-CEA;

FIG. 2.18 shows the detection limit of detection of CR-ovalbumin;

FIG. 2.19 illustrates the concentration limit of detection of CR-PSM;

FIG. 2.20 describes a washing experiment to remove bound glycoproteins;

FIG. 2.21 compares the washing and rebinding for the reuse of PBL library;

FIG. 2.22 illustrates a PBL library that bound different FITC-labeled glycoproteins;

FIG. 2.23 illustrates PBL arrays;

FIG. 2.24A is a schematic representation of a phenylboronic acid substituted peptide (PBL) binding to a glycan or glycoprotein;

FIG. 2.24B illustrates a biased split-and-pool method used to generate the ‘low’ diversity PBL library;

FIG. 2.25 illustrate microscope images of a PBL library binding to different glycoproteins, having the bound analyte washed away and showing rebinding. The lack of non-specific interactions is depicted in the last two rows with BSA and the PBL, and a glycoprotein with blank resin;

FIG. 2.26 illustrates microscope images of individual beads responding to FITC-labelled glycoproteins: showing representative selective (G2, E1), partially cross-reactive (H4) and completely cross-reactive (A6) library members.

FIG. 2.27 illustrates patterns from a micro-titer plate based PBL array responding to BSM (yellow), PSM (blue), Oval (red) and the composite response showing selective and cross-reactive PBLs;

FIG. 2.28 illustrates microscope images of the PBL library response to CEA at 10 ng/mL and 10 pg/mL;

Table 2.2 shows the comparison of addition of glycerol;

Table 2.3 shows the detection limits of a few fluorescently-labeled glycoproteins; and

Table 2.4 describes the binding sequence of PBL arrays as described herein.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

Generally speaking, novel peptide-based borono-lectin (“PBL”) sensors, along with their synthesis and analysis and methods of use, are described. These sensors use peptides as the scaffold and introduce boronic acid moieties onto the peptide scaffold as the binding site for the targeted analyte (e.g., carbohydrates, glycans, etc.). The synthesis of these PBL sensors can be higher yielding and cost-effective because of conventional peptide synthesis protocols. Additionally, formation of diverse sensors can be easily accomplished through changing amino acid monomers in the scaffold sequence and adjusting the length of side chains of peptides to offer numerous differences in the geometry, valency, and therefore binding affinity and selectivity of the sensors. Thus, numerous sugar specific and cross-reactive biosensors can be readily developed, which can establish PBL microarrays and enable the facile identification of the best sensors for specific target molecules. In another aspect, the employment of peptides as the sensor backbone addresses the problems of previously known materials associated with lower water solubility and biocompatibility of sensors based on organic compounds. Also, the use of PBL-based sensors can be quite safe because they are synthesized from nature's own building blocks, amino acids. In addition, there is no folding to be concerned about in the specific case of using short peptides rather than entire proteins as scaffold materials. Finally, the introduction of boronic acid functional groups can provide strong intermolecular interaction; thus, more stable sensors can be produced.

Presently disclosed are biosensors developed from Peptide-Based Borono-Lectins (PBLs) that are particularly suitable for detecting the presence of a targeted carbohydrate and/or glycoconjugate. Synthetic methodologies for generating PBLs are generally disclosed, including methods for the introduction of boronic acid moieties to peptide side chains and the generation of PBL library with a diversity of spatial orientation. Moreover, synthetic protocols to prepare the conjugates of fluorescent compounds and glycoproteins is also disclosed. An exemplary PBL library is also disclosed. It has been found that PBLs show selective and cross reactive interactions to targeted sugars and glycoproteins. A novel type of PBL sensors for carbohydrates and glycoproteins with good biological compatibility, reduced synthetic demand, high synthetic diversity, and low toxicity is introduced as a diagnostic tool.

The present disclosure, in one embodiment, features improved biosensor devices, and methods for using such biosensor devices for detecting and/or quantifying the presence of a carbohydrate or glycan of interest within an aqueous medium. The receptive material of disclosed biosensors includes a boronic acid functionality incorporated into a protein-based scaffold. In a particular embodiment, the boronic acid functionality is present on an amino acid in the protein-based scaffold, and the placement of these boronic acid functionality groups can be engineered to bond to specific carbohydrates or glycoproteins, effectively becoming a “synthetic lectin.” The amino acid(s) having the boronic acid functionality can be engineered to be the protein scaffold backbone and/or in an amino acid or a peptide side chain extending off of the peptide backbone. Generally, the placement of the boronic acid functionalities on the protein scaffold will vary according to the particularly targeted diol to be attached (e.g., the particularly targeted carbohydrate or glycoprotein).

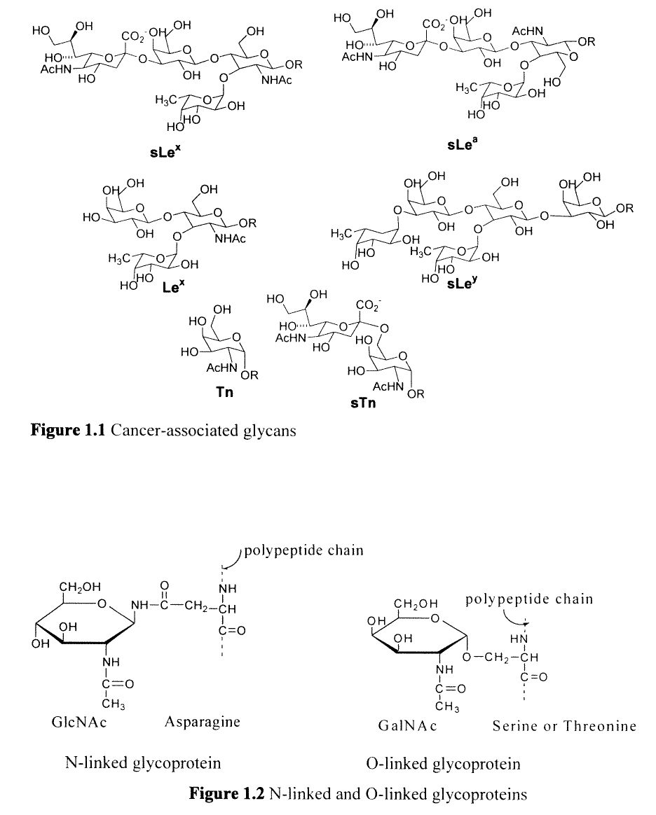

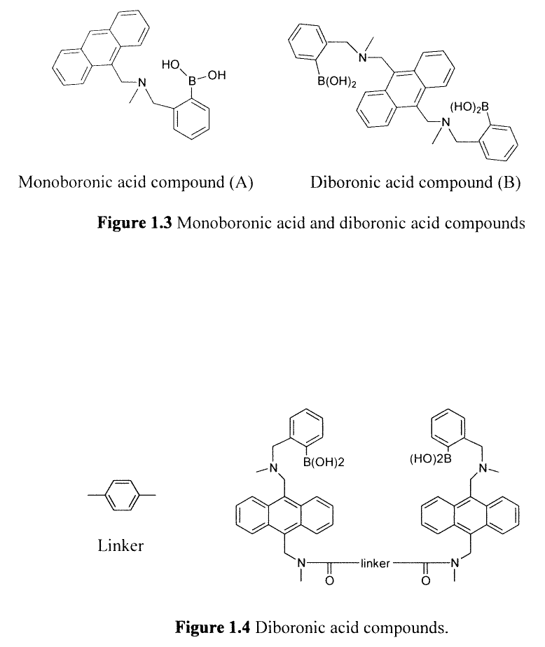

The present invention is also generally directed to a method to design, synthesize and analyze peptide-based borono-lectin sensors as diagnostic biosensors for carbohydrates and glycoconjugates as may be utilized in diagnosis or treatment of diverse diseases such as cancers, inflammation, and diabetes. Specifically, disclosed biosensors can detect the presence, or absence, of a carbohydrate or glycoconjugate that is known to be indicative of a particular disorder. For example, a biosensor can be used to detect the presence of the cancer-associated glycans of FIG. 1.1 to indicate that it is present in a sample. If the particular carbohydrate or glycoprotein is present in the sample, then the sample supplier can be diagnosed as having or potentially having a disorder associated with the targeted analyte and treated or tested further accordingly.

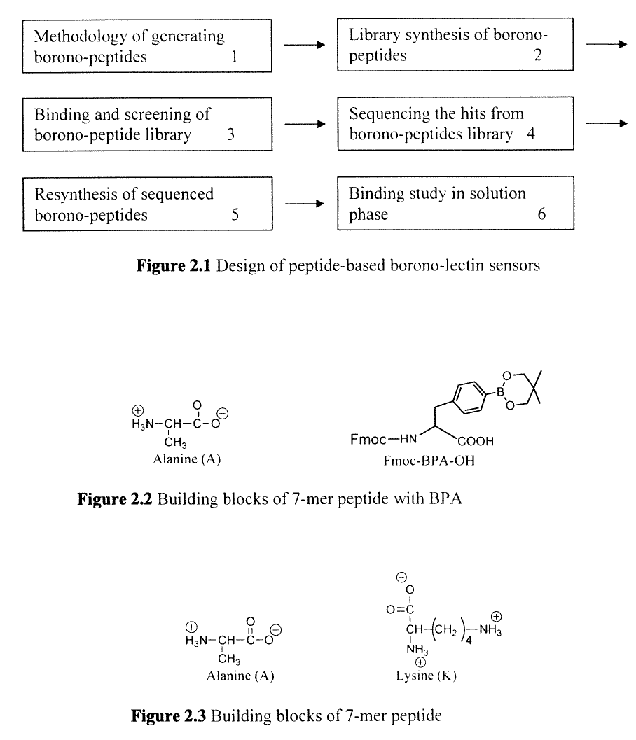

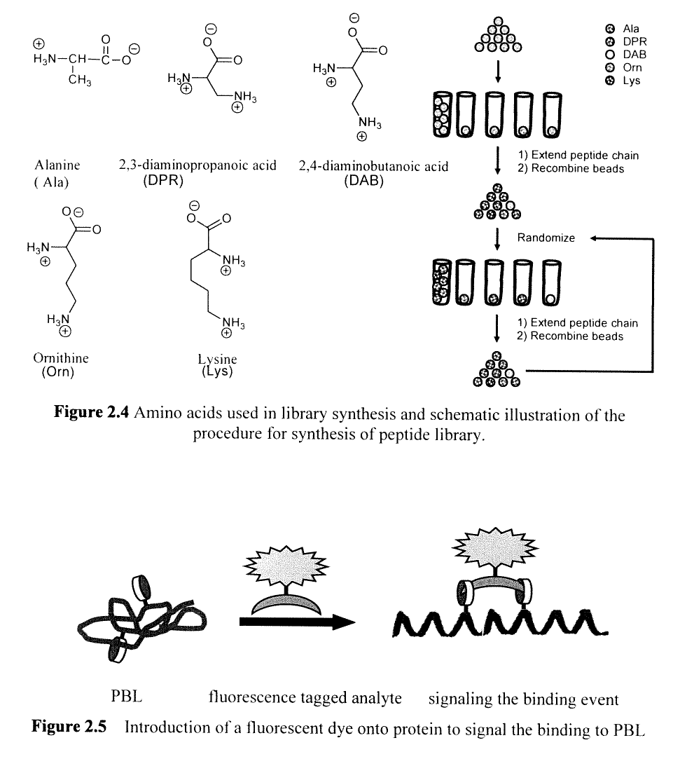

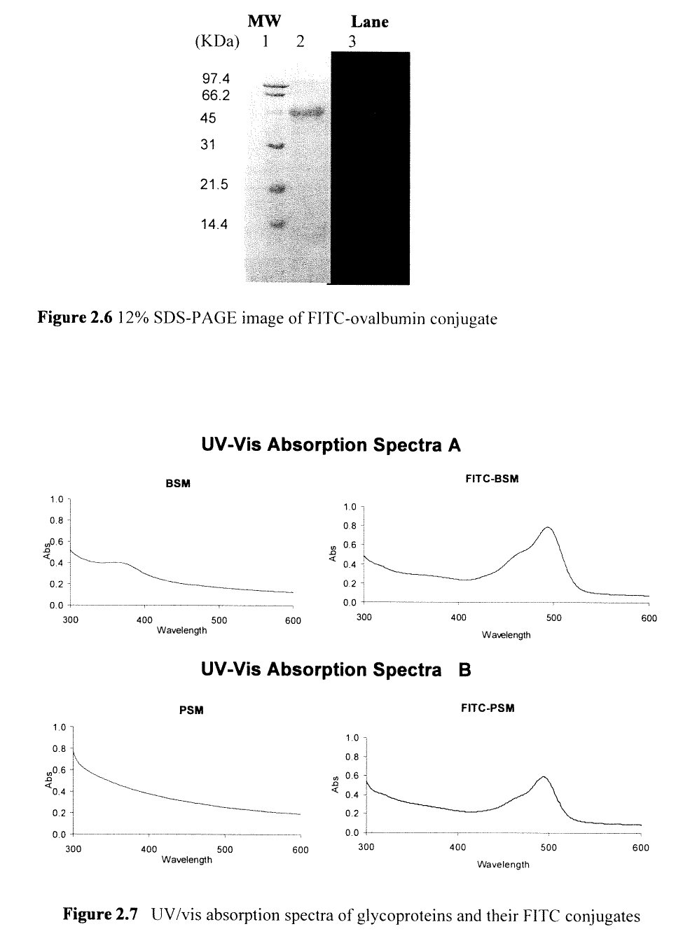

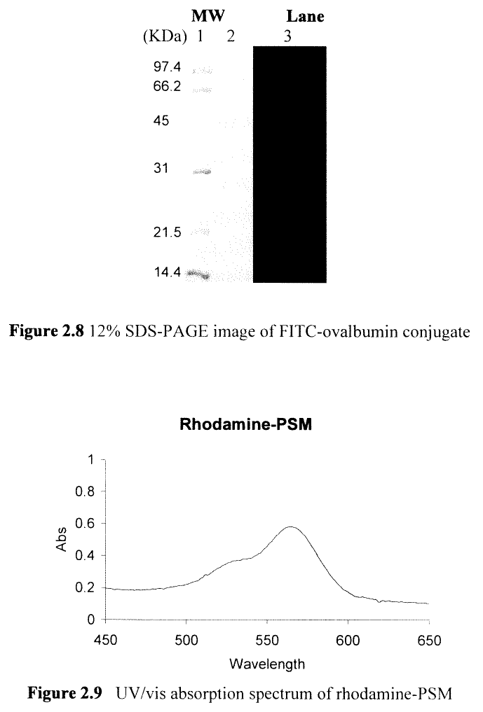

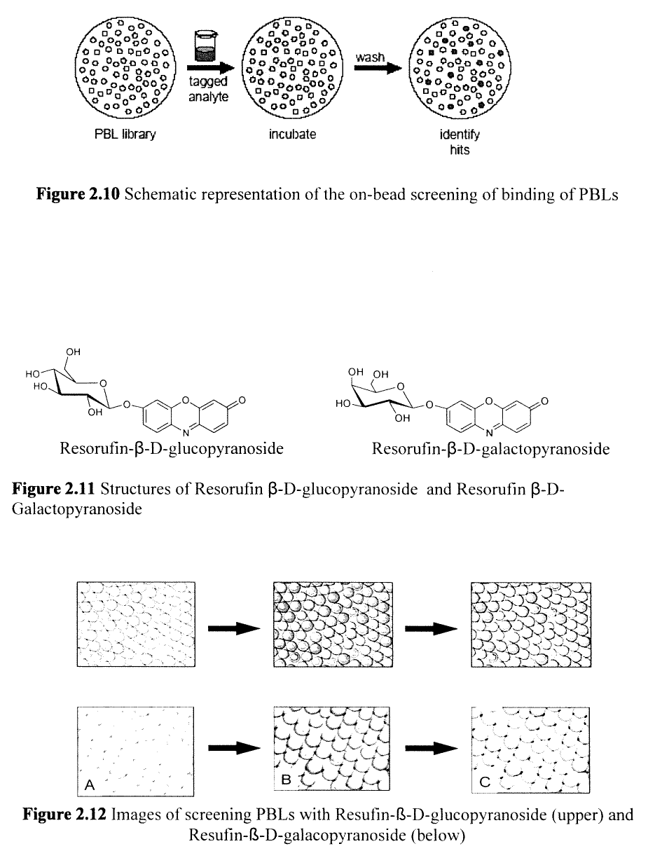

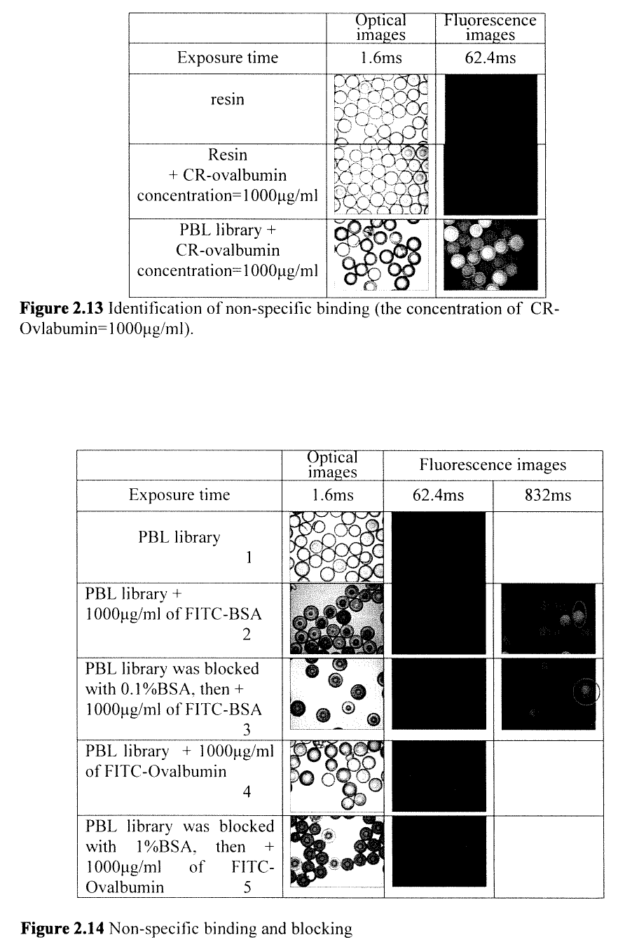

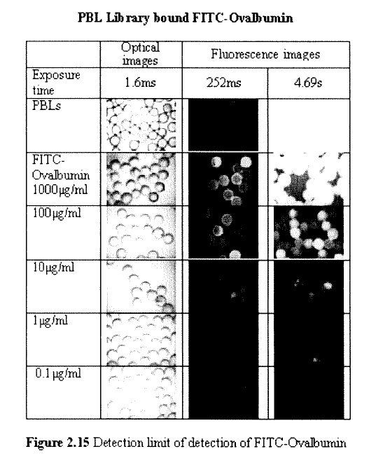

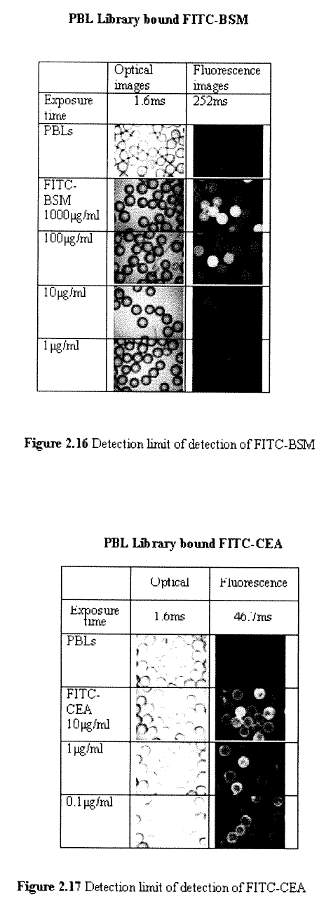

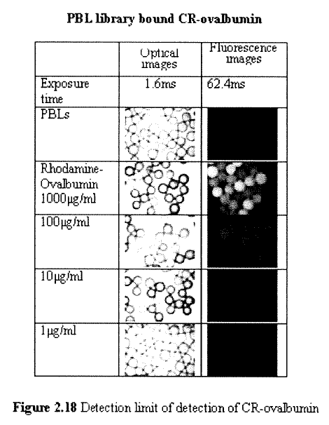

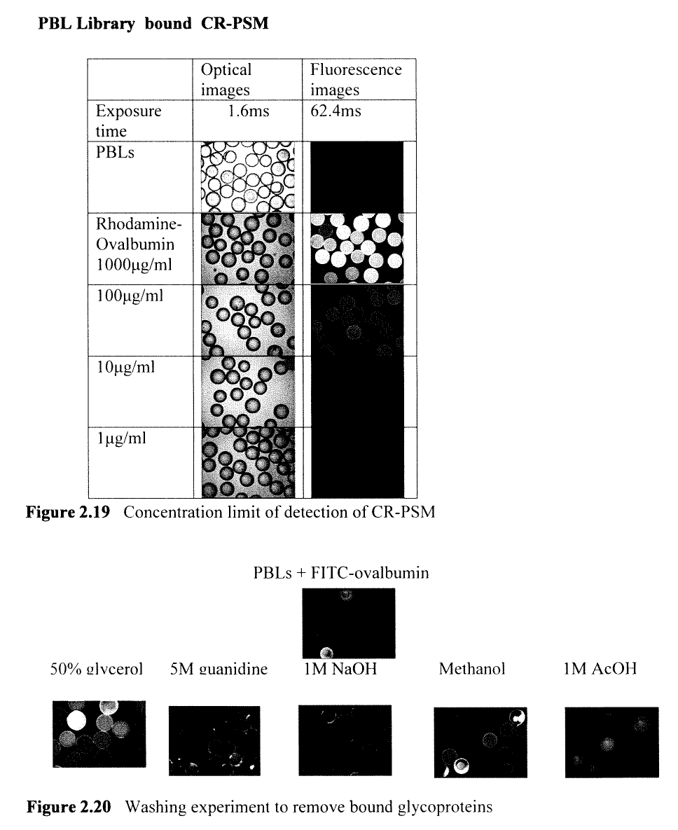

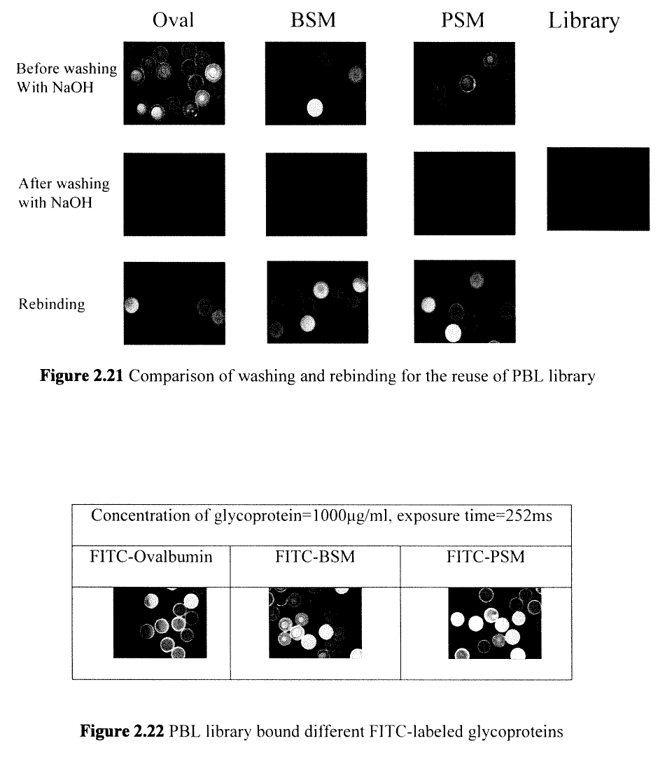

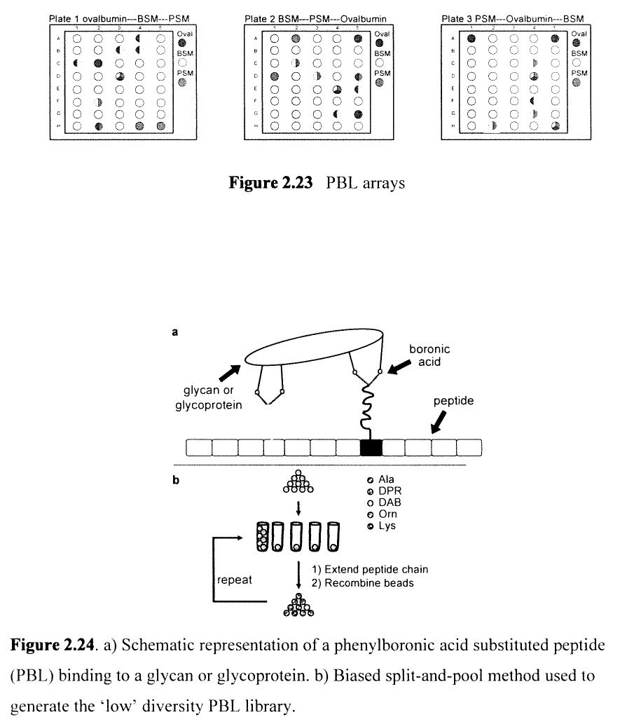

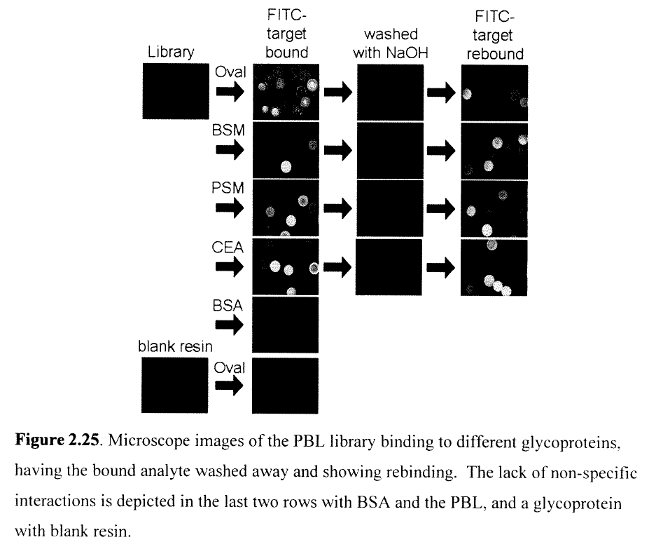

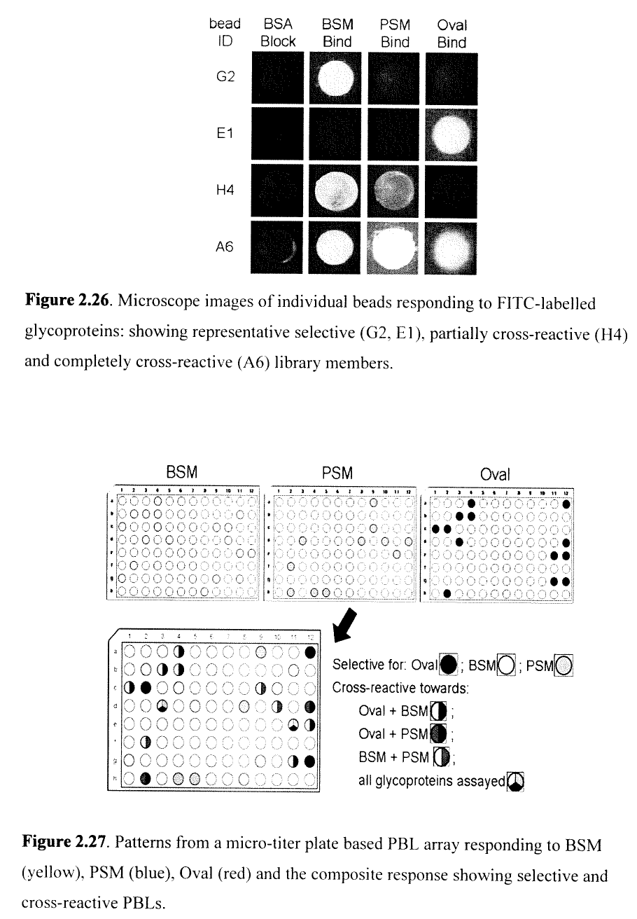

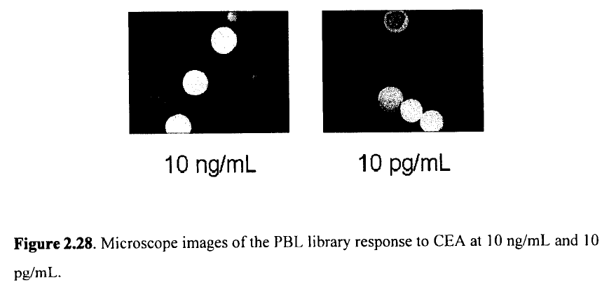

One embodiment of the disclosed subject matter is directed to the development of a BPL library that can be utilized to screen a sample for one or more glycans. One embodiment of developmental stages for preparing a BPL library are illustrated in the flow chart as shown in FIG. 2.1 and further described below.

Stage 1: Establishment of Methodology for Generating Borono-Peptides

Boronic acid moieties can be efficiently introduced onto peptide side-chains as the receptors of carbohydrates and glycoproteins. Generally, any boronic acid moieties that can be attached to an amino acid within a peptide can be included. For instance, one boric acid functionality can be attached to a single amino acid in the peptide scaffold as described herein.

The protein scaffold can be a relatively small peptide carrier to which one or more boronic acid functionalities can be grafted. When using relatively small peptides, the folding and unfolding nature of larger proteins can be avoided, while still providing a peptide based scaffold that is compatible with the test sample. For example, the protein scaffold can include relatively short peptides, such as those having less than 30 amino acids bonded together, such as from about 5 to about 25 amino acids. The protein scaffold of the disclosed devices can include at least one boronic acid binding site oriented in a manner to bond to a targeted carbohydrate or glycan.

A boronic acid moiety can be incorporated into the peptide scaffold, for instance into a side chain of the peptide scaffold, according to any suitable methodology. For instance, in one embodiment, the boronic acid can first be incorporated into an amino acid residue, which can then used to synthesize the desired peptides configured to bond to the analyte of interest. Alternatively, peptides with protected amino groups in their side chains can be synthesized first, and then boronic acids can be incorporated onto the peptides through reductive amination between the deprotected amino groups and 2-formylphenyl boronic acid. Both of these methods are described further in the example section, below.

Stage 2: Library Synthesis of Borono-Peptides

Following incorporation of the boronic acid functionality onto the side chains of the peptides, a borono-peptide library can be produced. A split-and-pool library synthetic approach, as is generally known in the art, can be utilized in one preferred embodiment, as a diverse compound pool can thus be obtained at one time and the formed library can provide a reusable source that enables the identification of selective peptide sequences. In order to obtain the borono-peptide library, first, a peptide backbone library can synthesized, for instance via a split-and-pool solid phase combinatorial approach. Five amino acids that are particularly useful for a peptide backbone library to construct a peptide library with the protection of the N-terminus by benzyloxycarbonyl (Cbz), include alanine (Ala), 2,3-diaminopropanoic acid (DPR), 2,4-diaminobutanoic acid (DAB), ornithine (Orn) and lysine (Lys). These amine-containing amino acids can be used to produce different lengths of side chains of peptides, since they contain a different number of methylene groups. Therefore, diversity of the peptide sequence provides variation in horizontal direction and diversity of length of side-chain in the vertical direction can be obtained. Before introduction of the boronic acids onto the side chains of the peptides, it is necessary to remove the Boc protecting groups on the side chain amines. Subsequently, peptides are converted to borono-peptides by reductive amination between the deprotected amino groups and 2-formylphenyl boronic acid. This diverse borono-peptide library is now ready for screening.

Stage 3: Binding and Screening of Borono-Peptide Library

The matrix or medium containing the analyte of interest (e.g., the particular carbohydrate) may be a liquid, a solid, or a gas, and can include a bodily fluid (e.g., mucous, saliva, urine, fecal material, tissue, marrow, cerebral spinal fluid, serum, plasma, whole blood, sputum, buffered solutions, extracted solutions, semen, vaginal secretions, pericardial, gastric, peritoneal, pleural, and the like). Generally, it is contemplated that the medium containing the analyte of interest be an aqueous based liquid solution, dispersion, mixture, or the like. For example, the medium can be an aqueous solution containing a tissue sample.

In one preferred embodiment, the sample to be tested can be fluorescently tagged. For example, the material in the sample can be reacted with a fluorescent reagent that adds a fluorescent characteristic to the material without substantially changing the chemical structure of the material. In one particular embodiment, for instance, the material with the sample can be tagged with a fluorescent tag as known in the art. The tag can be a fluorescent molecule (also known as a fluorophore), such as fluorescein and green fluorescent protein. For example, fluorescein isothiocyanate, a reactive derivative of fluorescein, can be utilized to tag the protein material in the sample. Other common fluorophores include derivatives of rhodamine, coumarin and cyanine. Of course, any fluorescent compound can be used to tag the material within the sample.

When the sample has been fluorescently tagged, any suitable energy source may be selected for irradiating the biosensor device to detect if the targeted analyte bonded to the boronic acid functional receptive material. The energy source may be, for example, a light source, e.g., an ultraviolet (UV) light source, an electron beam, a radiation source, etc. The invention is not limited to any particular wavelength of the UV light or exposure times. Wavelengths and exposure times may vary depending on the particular type of receptive material. Other suitable energy sources may include tuned lasers, electron beams, various types of radiation beams including gamma and X-ray sources, various intensities and wavelengths of light including light beams of sufficient magnitude at the microwave and below wavelengths, etc.

In one embodiment, the protein scaffold containing the boronic acid functionality can be attached to a particle that provides a structural support for the scaffold. For instance, the boronic acid functional protein scaffold can be disposed on “beads” or “microbeads”. Naturally occurring particles, such as nuclei, mycoplasma, plasmids, plastids, mammalian cells (e.g., erythrocyte ghosts), unicellular microorganisms (e.g., bacteria), polysaccharides (e.g., agarose), etc., may be used. Further, synthetic particles may also be utilized. Although any synthetic particle may be used in the present invention, the particles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. When utilized, the shape of the particles may generally vary. In one particular embodiment, for instance, the particles are spherical in shape. However, it should be understood that other shapes are also contemplated by the present invention, such as plates, rods, discs, bars, tubes, irregular shapes, etc. In addition, the size of the particles may also vary. An exemplary commercially available particle having an amino acid functionality includes Fmoc-Alanine-Wang resin, which has the structure below (where X is a halogen) and is provided on a polystyrene matrix having a particle size of from about 100 to about 200 (mesh).

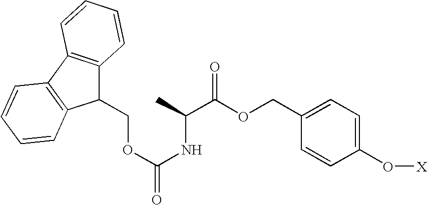

The particle, such as the Fmoc-Alanine-Wang resin, can be utilized as an anchor for the protein scaffold and can provide a structural support for the protein scaffold.

The protein scaffold can generally be attached to the particles or other surface using any of a variety of well-known techniques. For instance, covalent attachment of the protein scaffold to the particles can be accomplished using carboxylic, amino, aldehyde, bromoacetyl, iodoacetyl, thiol, epoxy and other reactive or linking functional groups, as well as residual free radicals and radical cations, through which a protein coupling reaction can be accomplished. A surface functional group can also be incorporated as a functionalized co-monomer because the surface of the microparticle can contain a relatively high surface concentration of polar groups. In addition, the particles may be capable of direct covalent linking with a protein without the need for further modification. Besides covalent bonding, other attachment techniques, such as physical adsorption, may also be utilized in the present invention.

The sample to be tested can then be contacted with (such as in a plate well, test tube, or other container) and incubated with fluorescently-tagged glycoproteins at room temperature for a time sufficient (e.g., 10 hours or greater) to bond any glycoproteins or carbohydrates. Then, the beads can be washed to remove unbound glycoproteins and carbohydrates. If the beads are fluorescent after washing, then a glycoprotein was bonded to the boronic acid moiety of the bead.

Binding is followed by screening with the aid of optical and fluorescence microscopies. Some individual PBLs show specific and strong binding to certain sugars. The brightest ones are the strongest binding. Other PBL members may not bind specifically to a specific sugar or glycoprotein, but they may show binding with multiple targets, i.e., they will represent cross-reactive PBLs. Ultimately, both types of PBLs can be used to generate sensors for a variety of sugars and glycoproteins.

Stage 4: Sequencing the Hits from Borono-Peptides Library

The sequences of both specific and cross reactive sugar binding borono-peptides can be determined following the deprotection of N-terminal Cbz groups.

Stage 5: Resynthesis of Sequenced Borono-Peptides in Solution Phase

The sequenced peptides can then be resynthesized by solid phase methodology. Then, the boronic acid moieties can be attached, and the PBLs cleaved from the resin to facilitate solution studies.

Stage 6: Binding Study in Solution Phase

Sugar and glycoprotein binding to specific and cross reactive PBLs can be investigated with the aid of NMR and CD. Also, the binding stoichiometry and affinity can be evaluated.

To demonstrate the stages described above, the following studies were carried out. These studies and the resulting discussion are intended merely to illustrate the invention and are not intended to limit the scope of the invention.

### EXAMPLE 1

### I. Synthesis of Borono-Peptide (Stage 1)

To complete the studies, the synthetic methodology for incorporating boronic acid moieties into peptides first needs to be established. Two routes were designed. The first route involved synthesizing Fmoc-boronophenylalanine (Fmoc-BPA) as a photoactivable amino acid monomer which is then incorporated with other amino acids to generate borono-peptides on alanine Wang resin using an automated solid-phase peptide synthesizer. The other route involved the incorporation of boronic acids through reductive amination using 2-formylphenylboronic acid and sodium borohydride after the peptide backbones were constructed.

A. Synthesis of Borono-Peptides Based on Fmoc-BPA

Commerically available boronophenylalanine (BPA) was protected as in Scheme 2.1 to generate Fmoc-BPA(Neo)-OH as a building block for use in an automated peptide synthesizer. The amino group was first protected with Fmoc-Cl (9-fluorenylmethoxycarbonylchloroformate) under standard conditions and the boronic acid was then protected via the formation of boronate ester with neopentyl glycol through a simple dehydration with the removal of water using a Dean-Stark trap.

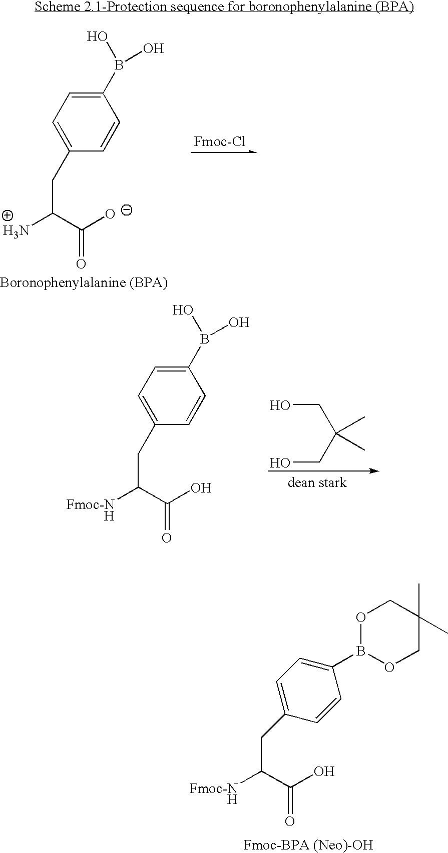

Subsequently, Fmoc-compatible solid phase synthesis of peptides on alanine Wang resin using alanine and Fmc-BPA(Neo)-OH as building blocks (FIG. 2.2) was conducted and the 7-mer peptide Ac-A-A-A-BPA-A-BPA-A was fabricated. After synthesis, the peptide was cleaved from the resin using Reagent K (760 mg phenol, double distilled-water 750 μl, thioanisole 750 μl, 1,2-ethanedithiol 375 μl, and TFA 28.5 ml, TFA is about 95%). However, the MS-ESI analysis indicated that partial hydrolysis of the boronate to the phenol had occurred during cleavage from the resin. Because of these synthetic challenges and the fact that BPA is expensive, an alternative route was adopted for incorporating the phenyl boronic acid moiety onto peptides.

B. Incorporation of Boronic Acid into Peptides through Reductive Amination

There are at least three intrinsic benefits of incorporating the phenyl boronic acid moiety through the reductive amination of an orthogonally protected diamino acid residue, e.g, lysine, using 2-formylphenylboronic acid as the boronic acid-containing moiety. First, the commercially available reagents used in this reaction are inexpensive. Second, the presence of nitrogen as a Lewis base nearby to the boron enhances the binding interactions with sugars compared to the simple phenylboronic acids moiety found in BPA, which results in more stable binding. Finally, the distance between the boronic acid and peptide backbone can be varied by varying the number of methylenes joining the two. This allows for additional control over the geometric placement of the boronic acid groups.

To develop the synthetic methodologies to incorporate the phenylboronic acid moiety into peptides adopting the second synthetic route, model reaction 1 (scheme 2.2) was designed to validate reductive amination in solution phase. Mass spectrometry showed that the model reaction worked well and it provided a reference for the synthesis of borono-peptides.

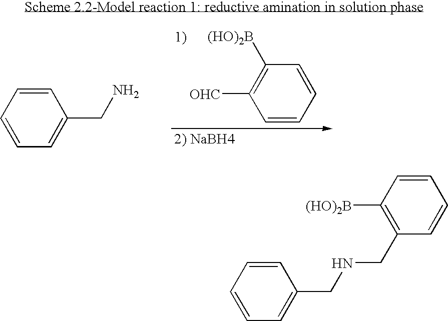

Subsequently, three 7-mer peptides (Ac-A-A-A-K-A-K-A, Ac-A-A-K-A-A-K-A, and Ac-A-K-A-A-A-K-A) were synthesized on alanine-Wang resin using the building blocks shown in FIG. 2.3. Briefly, peptides were synthesized using a PS3 automated peptide synthesizer and involved the sequential couplings of lysine (K) or alanine (A) based on the sequence of the three peptides. The peptides were cleaved from resin using Reagent K. The Boc protection groups on the lysine residues were deprotected simultaneously using these conditions. Finally, as described above, phenyl boronic acids were introduced into peptides through reductive amination with 2-formylphenylboronic acid and sodium borohydride in methanol (Scheme 2.3). Crude products were purified by HPLC and clean diboronopeptide obtained with excellent yield (84.5%).

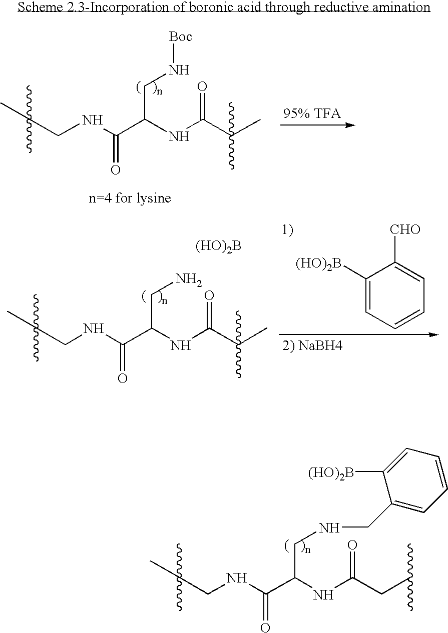

### EXAMPLE 2

### Synthesis of Borono-Peptide Library (Stage 2)

To obtain a borono-peptide library to provide as many candidates as possible for screening, two steps needed to be completed. First, a 12-mer peptide library was generated via the split-and-pool combinatorial solid phase synthesis approach. Second, phenyl boronic acid moieties were introduced by the reductive amination method described previously.

The formation of a peptide library and amino acid monomers used are illustrated in FIG. 2.4. Five amino acid monomers, Ala, DPR, DAB, Orn and Lys, were used with the ratio of 6:1:1:1:1 to produce a relative low diversity library. The theoretical diversity of the resulting PBL library is on the order of 10 million distinct peptide sequences containing a statistical average of 4 PBA moieties per peptide. Because only 1 g of resin was used in the construction of this library, the number of unique PBL sequences is on the order of 2,000,000. The combination of these four amine-containing amino acids with a different number of methylene groups to the amine can give a diverse spatial arrangement of the boronic acid and therefore create a diverse group for the borono-peptide library, which is beneficial for selective carbohydrate and glycoprotein binding.

The first alanine was attached to Nova TG aminoethyl PEG-PS resin (110 μm) which is stable to acid hydrolysis to give a starting point for library synthesis. The resin was then split into 10 equivalent portions, which were divided into five groups with the ratio 6:1:1:1:1. Then, the five groups were coupled separately with Fmoc-Ala-OH, Fmoc-DPR(Boc)-OH, Fmoc-DAB(Boc)-OH, Fmoc-Orn(Boc)-OH, and Fmoc-lys(Boc)-OH. All the beads were combined and the same procedure was repeated 9 times. Finally, a (Cbz)-Ala-OH residue was added to the terminal amine and the 12-mer peptide library: (Cbz)-A-(X₁₀)-A-resin was obtained. Here X is one of five amino acids shown in FIG. 2.4. Cbz-Ala-OH was incorporated at the N-terminus of the peptide to allow for selective deprotection of the α-amino group, thereby permitting the identification of the peptide sequence by Edman degradation. The Boc protecting groups were then removed with 95% TFA and free amino groups were obtained for incorporation of boronic acid moieties.

### Synthesis of Borono-Peptide Library

Before incorporating boronic acids into the peptide library, the model reaction 2 shown in Scheme 2.4 was done. The reductive amination mentioned previously was carried out in methanol but the polymeric beads of the library did not swell well in such a polar protic solvent. Considering the need of both the reaction and swelling of resin, a mixed solvent was chosen. To test the mixed solvent effect, two solvent systems 100% methanol and mixed solvent (DMF/methanol: 9/1, v/v) were used for comparison. The reaction was designed to synthesize a simple borono-tripeptide: Cbz-Ala-Lys(PBA)-Ala-NH₂ (PBA is phenyl boronic acid). Thus, Fmoc-Ala-OH, Fmoc-Lys(ivDde)-OH, and Cbz-Ala-OH were used as building blocks and coupled to a Rink-AM resin in order (scheme 2.4). We chose the ivDde protecting group instead of a Boc group because its deprotection condition is orthogonal to the cleavage condition of Rink-AM resin. The Rink-AM resin was used due to its easy cleavage as the peptide must be cleaved from the resin for analysis. The ivDde group was removed using 2% hydrazine in DMF and the primary amine on lysine residue coupled to 2-formylphenylboronic acid by reductive amination using sodium borohydride to generate the borono-tripeptide on resin. Subsequently, the peptide was cleaved from Rink resin using 95% TFA and 2.5% TIS and 2.5% H₂O.

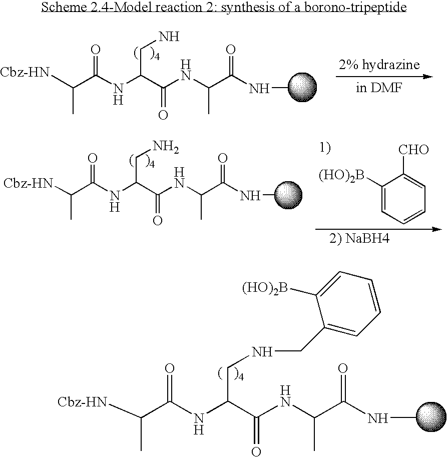

The samples resulting from the syntheses in the two solvent systems were analyzed by HPLC-mass spectrometry. The sample prepared with 100% methanol showed incomplete coupling. The sample from the mixed solvent system was found to afford near quantitative coupling of boronic acid to peptide; the corresponding molecular weight in HPLC-Mass-ESI was consistent with the calculated value. C₂₇O₇N₅H₃₈B, (M⁺+H) Calcd. 556.43; found 556. With the successful incorporation of phenyl boronic acid into a tripeptide on the solid phase, we extended our method to produce the borono-peptide library.

The boronic acids were incorporated into the peptide library by reductive amination between the primary amines of the side chain of peptide and 2-formylphenylboronic acid using the described mixed solvent system. The resultant borono-peptide library is diverse both in sequence and in spatial arrangement of side chains due to the different number of methylene units from DPR, DAB, Orn, and Lysine. Therefore, a pool of borono-peptides was generated for screening.

### EXAMPLE 3

The binding events can be signaled through the fluorescence off-on produced by certain fluorescence compounds upon binding. However, for the screening of the PBL library conducted currently, a simple and easy method involving directly labeling glycoproteins with a fluorescent dye such as fluorescein isothiocyanate (FITC) or carboxyrhodamine (CR) was used, thus the appearance of fluorescence will signal the binding between PBLs and glycoproteins, as illustrated in FIG. 2.5. This signaling method is just used for screening of the PBL library.

As a preliminary step, we labeled the four glycoproteins listed in Table 1.2: Ovalbumin (Oval), Bovine Submaxillary Mucin (BSM), Porcine Stomach Mucin (PSM) and Carcinoembryonic antigen (CEA). These glycoproteins are varied in molecular weight, amount and the type of carbohydrates incorporated, as shown in Table 1.2:

TABLE 1.2
Comparison of ovalbumin, BSM, PSM and CEA

| | Molecular | | |
|---|---|---|---|
| Proteins | Weight | Source | Associated Carbohydrates |
| Oval | 45 KDa | Chicken egg white | Mannose, N- |
| | | | acetylglucosamine |
| BSM | 400 KDa | Submaxillary gland | Sialic acid, N- |
| | | of bovine | acetylgalactosamine, |
| | | | galactose |
| | | | N-acetylglucosamine, |
| | | | fucose |
| PSM | 1000 KDa | Stomach of pig | Similar to bovine |
| | | | submaxillary mucin |
| CEA | 200 KDa | Human fluid | Fucose, sialic acid, |
| | | | galactose, mannose, N- |
| | | | acetylglucosamine |

Ovalbumin, which is extracted from chicken egg white, is a relatively smaller N-linked glycoprotein (M.W. 45 KDa). In structure, it has mannose and N-Acetylglucosamine. The content of carbohydrates is 3.2% by wt of protein. Mucins are glycoproteins with more than 50% oligosaccharides which are attached to serines and threonines via O-linkages. The main functions of mucins are lubrication and protection due to their high viscosity. For instance, salivary mucins play a role in lubrication during swallowing. Mucin molecules produced by cells of gastrointestinal, respiratory and genitourinary tracts can protect the epithelium. It has been reported that BSM has sialic acid, N-acetylgalactosamine, N-acetylglucosamine, galactose, and fucose and the content of carbohydrates is 56.7% by wt of protein. Its molecular weight is 400 KDa. PSM has a molecular weight of 1000 KDa and its content of carbohydrates is 76±20% by wt of protein. Carcinoembryonic antigen, an apical membrane glycoprotein expressed in normal human colonic epithelial cells, colonic polyps, tumor, and tissue culture cell lines originating from colonic adenocarcinomas, is generally considered to have a molecular weight of 200 KDa and carbohydrate composition is about 50-80%. These compounds are model glycoproteons for two reasons. First, these glycoproteins contain carbohydrates and some of these carbohydrates are cancer-related. For example, it has been found that increased expression of several carbohydrates such as sLe^{x} and sLe^{a}, has been correlated in colon, breast, gastric, pancreatic, and lung cancer. Second, the synthesis of complex carbohydrates is not easy and thus these natural glycoproteins can serve as ideal mimics. In addition, it has been reported that PSM is similar to human tracheal mucin in its carbohydrates.

We first synthesized the conjugate of fluorescein isothiocyanate-ovalbumin, so-called FITC-labeled ovalbumin. Referring to the literature, we incubated commercially available chicken ovalbumin with FITC in carbonate buffer (pH 9.5) for 1 hour at room temperature. Subsequently, the solution was dialyzed against phosphate buffered saline solution (PBS, pH 7.3). The purity of the conjugate was assessed with Sodium Dodecyl Sulfate-Polyacrylamide Gel Electrophoresis (SDS-PAGE). As shown in FIG. 2.6, the fluorescent band on the right and the Coomassie Blue stained band in the left show the molecular weight is 45 KDa which is consistent with the reported molecular weight of ovalbumin. Therefore, the formation of a FITC-ovalbumin conjugate was confirmed.

Similarly, the conjugates of FITC with Bovine Submaxillary Mucin (BSM), and Porcine Stomach Mucin (PSM) were also synthesized. Due to the larger molecular weights of both conjugates (BSM=400 KD, PSM=1000 KD), they remained in the stacking gel and did not move in the gel when SDS-PAGE analysis was performed. Therefore, we adopted UV/vis absorption spectroscopy to characterize the formation of these conjugates. FIG. 2.7 shows the UV/vis absorption spectra A for BSM and its FITC conjugate and spectra B for PSM and its FITC conjugate. It can be seen that the products of the conjugation have the characteristic absorption of FITC (excitation wavelength around 494 nm) that was not observed for the unlabeled starting material, which confirms the formation of the conjugates. Finally, we also labeled CEA with FITC using the same protocol as described above.

Ovalbumin and PSM were also labeled with carboxyrhodamine. Carboxyrhodamine was first converted to its N-hydroxysuccinimide ester prior to its use. Then the rhodamine N-hydroxysuccinimide ester was incubated with ovalbumin or PSM in 0.1M carbonate buffer (pH=8.5) at 28° C. for 1 h. Subsequent dialysis was conducted (0.1M carbonate buffer, pH=8.5 followed by PBS). The SDS-PAGE image shown in FIG. 2.8 indicates the formation of the conjugate between rhodamine and ovalbumin (MW=45 KDa). Similarly, the UV/vis absorption spectroscopy was also used to characterize the formation of the conjugate of carboxyrhodamine N-hydroxysuccinimide ester and PSM, as shown in FIG. 2.9.

### IV. Binding and Screening of Borono-Peptide Library(Stage 3)

The PBL library was screened with simple sugars and complex glycoproteins. In order to facilitate the screening, dye-tagged sugars or fluorescently-tagged glycoproteins were employed to signal the binding with resin bound borono-peptides. For specific sugar binding borono-peptides, obvious changes in the color of the beads could be seen with optical microscopy when color-tagged sugars were used; for fluorescently tagged analytes, strong fluorescence could be observed by the aid of a fluorescence microscope. FIG. 2.10 shows the basic principle of the library screening and hit identification. Generally, the borono-peptide library is incubated with tagged analytes overnight and the beads are washed by PBS several times to remove unbound protein. When the library is screened under an optical or fluorescence microscope, hits (bright colored or fluorescent beads) can be identified and sorted out.

Binding and Screening of PBL Library with Simple Sugars

To validate our strategies to identify individual PBLs that preferentially bind to carbohydrates and glycoproteins, we initially focused on identifying PBLs that demonstrated selective binding for either glucose or galactose because these two simple sugars are isomers and display differential stereochemistry at only one position. To visually observe the binding event, commercially available resorufin tagged glucose and galactose (resufin-β-D-glucopyranoside and resufin-β-D-galacopyranoside) were used. The structures of these two dye-tagged simple sugars are shown in FIG. 2.11.

The color-tagged sugars were dissolved in a small amount of DMF first and then diluted with Phosphate-Buffered Saline (PBS) buffer. The PBL library was then incubated with sugar solution at ambient temperature for 16 hours. After washing the PBL library 3 times with PBS buffer to remove unbound sugars, the ability of the PBLs to bind the dye-tagged sugars was assessed with an optical microscope. Colored beads signal the binding between sugars and borono-peptides. The upper row in FIG. 2.12 represents the PBL library bound with Resufin-β-D-galactopyranoside and the lower row is the PBL library bound with Resufin-β-D-glucopyranoside. Image A is the original image, B is after background has been subtracted and C is the images filtered through a red channel. The results of these studies indicate that even with a PBL library of high alanine content there exist galactose specific PBL sequences.

Binding and Screening of PBL Library with Glycoproteins

With the FITC or CR tagged glycoproteins in hand (Section 2.3.3), we used them to screen the library and qualitatively identify the PBLs that selectively bind or have a cross-reactive interaction with these fluorescently labeled glycoproteins. The general procedure of binding is that the PBL library is incubated with fluorescently-tagged glycoproteins at room temperature overnight and then the beads are washed using PBS buffer to remove unbound glycoproteins. For the binding of the PBL library to CR-glycoproteins, after binding, the library beads are washed with methanol first to remove trace amounts of rhodamine impurities and followed by PBS washing. Then fluorescence images are taken with a fluorescence microscope.

To evaluate whether there was non-specific binding, we used the resin to bind CR-ovalbumin and the images are shown in FIG. 2.13. Compared with resin, the CR- ovalbumin bound resin looks light red, which indicates there exists some non-specific binding, but it is minimal when compared to the amount of CR-glycoprotein's specific binding.

On the other hand, we used the PBL library to bind FITC labeled bovine serum album (BSA) to observe non-specific binding. There should not be binding between them because there are no carbohydrates on BSA. The experimental results were compared in FIG. 2.14. Comparing 1 and 2 under exposure time 62.4 ms, non-specific binding is minimal because there is no significant fluorescence observed from 2. However, when PBLs bound FITC-ovalbumin (image 4 in FIG. 2.14), fluorescence can be observed clearly compared to 2. That means the amount of non-specific binding is less than specific binding. In order to see whether the blocking PBL library with BSA is useful to prevent the non-specific binding, we compared the binding samples with and without blocking with BSA. Two fluorescently labeled proteins were used: FITC-BSA and FITC-Ovalbumin. In FIG. 2.14, it is not easy to differentiate the images 2 and 3 under 62.4 ms exposure time. We increased the exposure time to 832 ms and compared quantitatively the luminosity of fluorescent beads (with red circles). The mean of luminosity for image 2 is 63.57 and 38.84 for image 3; while for image 4 and 5, they are 38.63 and 19.86 respectively. This indicates that blocking the PBLs with BSA is helpful to decrease non-specific binding. In one experiment (Table 2.2, row 1 and 4), we counted the rate of hits which decreased when the PBLs were blocked with BSA.

In order to identify only the strongest binders, we introduced another diol containing compound, glycerol, to compete for binding with glycoproteins. The results listed in Table 2.2 (row 1, row 2 and 3) indicate that the addition of glycerol is an effective method to decrease the amount of hits and thus facilitate the identification of the stronger PBL binders. On the other hand, if more glycerol (10%) was added, the effect was better. Comparing two fluorescence images (row 2 and row 3), we found both the fluorescence and the rate of the hits decreased when 10% glycerol was added. In later assays, we added 10% (v/v) glycerol into the fluorescently labeled glycoprotein solution.

The fluorescently-labeled glycoproteins we chose for this experiment are listed in Table 2.3. To determine how low the concentration is at which we can still differentiate the fluorescence of the beads, the PBL library beads were blocked with 1% BSA and then incubated with varying concentration of FITC-labeled ovalbumin, PSM and CEA and CR-labeled ovalbumin and PSM with the addition of 10% (v/v) glycerol overnight. After washing away unbound glycoproteins, fluorescence images were obtained. The PBL library without addition of glycoprotein was used as a control sample. Here, the limit of glycoprotein detection is defined as the lowest concentration of fluorescently-labeled glycoproteins at which their binding to PBL library beads can give enough fluorescence intensity for visual observation. However, the concentration limits are also exposure time dependent, e.g., for FITC-Ovalbumin, and FITC-BSM, the lowest concentrations for visual observation of fluorescence under exposure time 252 ms are about 10 μg/ml, but if the exposure time was increased, the fluorescence can be observed even under dilute concentration. For instance, for FITC-Ovalbumin, the detection limit will lower to about 0.1 μg/ml when exposure time is increased to 4.69 s (FIGS. 2.15, 2.16 and Table 2.3). For FITC-CEA, if observed under 252 ms, the fluorescence of beads is strong. Its concentration limit is about 0.1 (μg/ml) under this exposure time (FIG. 2.17). Stronger fluorescence from FITC-CEA if under the same exposure time as others may be due to CEA having more terminal glycans compared to others.

For CR-glycoproteins, since the PBL library itself is lightly fluorescent under excitation wavelength=510-560 nm, in order to avoid high background interference, we chose comparatively lower exposure time (62.4 ms) when we took the images (FIGS. 2.18 and 2.19). Under this exposure time, the concentration limits for CR-Ovalbumin and CR-PSM were 1 μg/ml (Table 2.3). If we mention the concentration limit of screening, we mention exposure time because the concentration limit is exposure dependent.

It is important to be able to reuse the PBL library. There are three reasons to reuse the PBL library. First, the individual peptide sequence on a bead might be able to bind more than one glycoprotein, which is called cross-reactive interaction. If bound glycoproteins can be washed away and rebound with another glycoprotein, we can reuse the PBL library to investigate cross-reactive interaction to find patterns. Second, if reusing the PBL library, we can find the selectivity of the PBLs. Third, the reuse of the PBL library can reduce synthetic cost and save time. The basic idea is that the bound glycoproteins could be removed from boronic acids leaving the recycled PBLs, which are ready to rebind. In an effort to achieve this goal, various chemicals such as glycerol, guanidine, NaOH, MeOH and HOAc were used (FIG. 2.20). The top image is PBLs binding FITC-Ovalbumin. These samples were then washed using the solutions in the second row overnight. Compared with the other four chemicals, NaOH worked best in this study.

Subsequently, the FITC-BSM and FITC-PSM was washed away with 1M NaOH. FIG. 2.21 shows the comparison of before and after washing for three different glycoproteins and their rebinding to PBLs. Choosing the PBL library as a control sample, we can see the recycled PBL library shows almost no fluorescence. However, when the beads are reincubated with fluorescently-labeled glycoproteins, the fluorescence reappears. Therefore, the recycled PBL library could be reused for binding.

We have observed that the PBL library can reveal different binding by showing a varying degree of fluorescence when binding glycoproteins. (FIG. 2.22). For the binding of PBLs, there may be two common scenarios. First, an individual PBL may only bind to one specific glycoprotein, which is termed a selective PBL. The second scenario that may occur is that an individual PBL may bind more than one glycoprotein, i.e., cross reactive PBL. However, we cannot be sure whether certain beads showing bright fluorescence in these images are selective binding for a certain glycoprotein because the beads move around and it is impossible to follow one bead exposed to different analytes. A better way to assess the selectivity and the cross-reactive interaction is to design an array of PBLs to hold the beads separately and sequentially assay the interactions.

To assess the selectivity and cross-reactivity of the PBL library, PBL beads were placed into three microtiter plates with one bead per well. The beads in each plate were sequentially bound with different glycoproteins. The binding sequence is described in Table 2.4. Plate 1 started from ovalbumin, plate 2 started from BSM and plate 3 started from PSM. After binding each protein, the beads in the plates were washed with 1M NaOH and PBS and then rebound with the second glycoprotein. The same procedure was followed for the third glycoprotein. We recorded the positions of bright beads depicted in FIG. 2.23.

In FIG. 2.23, a solid color represents that the sequence responded to only one glycoprotein, a so-called selective interaction. The combination of two or three colors means that the sequence responds to more than one glycoprotein. Non-color circles represent unbound or missing beads during the washing. For those beads showing selective binding, their sequences are decoded and then the peptide resynthesized to further investigate binding in solution. For the beads showing cross reactive interactions, they are resynthesized and organized into a specific array. The arrays show specific binding patterns when they bind different glycoproteins.

### Experimental Section

1. Preparation of Borono-Phenylalanine (BPA) Ester

a. Protection of Amino Group of BPA

Fmoc-Cl (9-fluorenylmethoxycarbonylchloroformate) (1.2 g, 4.55 mmol) was dissolved in 15 ml dioxane and borono-phenylalanine (BPA, 0.95 g, 4.52 mmol) was dissolved in 45 ml 10% (w/w) Na₂CO_{3.} The Fmoc-Cl solution was added dropwise into the BPA solution. Reaction was conducted in an icy water bath. After 1 h, the icy water bath was removed and the reaction continued for 2 h at ambient temperature. Then the solution was diluted with 60 ml water. The mixture was washed with ether two times (20 ml each) and the aqueous phase was adjusted to pH=1 with 37% hydrochloric acid. The acidic aqueous layer was extracted with ethyl acetate three times (20 ml each). The organic layer was combined and evaporated under reduced pressure to remove solvent. The resultant product was dried under vacuum overnight and the yield was 35%.

¹H NMR (300 MHz, CD₃OD) δ 7.28(d, 2H), 7.24 (d, 2H), 7.77(d, 2H), 7.57(d, 2H), 7.50(t, 2H), 7.35(t, 2H), 4.30(t, H), 4.55 (d, 2H), 2.90(d, 2H), 4.42(q, H).

b. Protection of Boronic Acid Group of BPA

Fmoc-boronophenylalanine (0.67 g, 1.55 mmol) and neopentyl glycol (0.16 g, 1.55 mmol) were added into 50 ml toluene. 10 ml MeOH was then added and the solution was refluxed overnight. Water was removed using 4 Å molecular sieves in the Dean-Stark apparatus. The remaining solvent was evaporated under reduced pressure and dried under vacuum. The foam-like product was dried under vacuum overnight and obtained in near quantitative yield.

2. Synthesis of Borono-Peptide: Ac-Ala-Ala-Ala-BPA-Ala-BPA-Ala

Weighed out Fmoc-Ala-Wang resin (0.68 mmol/g, 147 mg, 0.1 mmol) and added to the reaction vessel of PS3 Automated Solid Phase Peptide Synthesizer. Fmoc-Ala-OH (125 mg), Fmoc-BPA-OH (borono-phenylalanine, 195 mg), HBTU (152 mg) and HOBT (54 mg) were weighed respectively into different plastic vials. Acetic anhydride 50 μl was added into the last plastic vial. The synthesis was completed using Fmoc system (0.4 M N-methylmorpholine in DMF and 20% (v/v) piperidine in DMF acted as the activator and deprotection agent respectively.) in PS3 Automated Solid Phase Peptide Synthesizer. Upon completion, the resin was washed with DMF, EtOH, and CH₂Cl₂ (3×15 ml) and dried under water aspirator. Then peptides was cleaved from resin using 95% TFA (760 mg phenol, double distilled-water 750 μl, thioanisole 750 μl, 1,2-ethanedithiol 375 μl, and TFA 28.5 ml) for 2 h. Cold diethyl ether was then added into resulting solution and centrifuged for 10 min. The precipitate was washed with diethyl ether three times. The product was dried gently with N₂. Then double distilled-water was added and the solution was swiftly frozen in liquid N₂. Finally the product was dried using lyophilize overnight and white cotton-like product was obtained. Mass Spectrometry showed that borono-esters hydrolyzed to boronic acids and one phenyl boronic acid decompose to phenol.

MS-ESI+ calcd for C₃₅O₁₂N₇H₄₈B₁ (M⁺+H) 770.69; found 770. (One phenylboronic acid turned to be phenol); calcd for C₃₅O₁₃N₇H₄₉B₂ (M⁺+H) 798.69; found 798 (two phenylboronic acids).

3. Synthesis of Borono-Peptides Using Reductive Amination Method

a. Synthesis of 7-mer Peptide Backbones

With the same procedure described in 2.5.2, three 7-mer peptides were synthesized on 0.2 mmol scale and then cleaved from the resin using 95% TFA. The desired peptides were purified by preparative HPLC using a H₂O/Acetonitrile/0.05% TFA solvent system.

Fmoc-Ala-Wang resin(0.68 mmol/g) 294 mg, Fmoc-Ala-OH: 249 mg, Fmoc-Lys(Boc)-OH: 375 mg, HOBT: 108 mg, HUBT: 303 mg, acetic anhydride 50 μl.

Ac-A-A-A-K-A-K-A (Pure product: 0.0994 g, yield 74%)

Ac-A-A-K-A-A-K-A (pure product: 0.1178 g, yield 84%)

Ac-A-K-A-A-A-K-A (pure product: 0.1103 g, yield 82%)

MS-ESI calcd for each C₂₉O₉N₉H₃₅ (M⁺+H) 672.79; found 672.

b. Model Reaction 1: Reductive Amination

2-formylphenylboric acid (0.1439 g, 0.9597 mmol) was dissolved in 20 ml dry MeOH. 0.1 ml benzylamine (0.0981 g, 0.9154 mmol) and several molecular sieves were then added to the solution. After 4 h, NaBH₄ (0.0626 g, 1.831 mmol) was added and reacted for 1 h at room temperature. Solvent was evaporated under reduced pressure. In order to remove side product produced by reductive animation, 1% HOAc solution was added and stirred for 15 min and then water was removed with a lyophilizer. Then trimethylorthoformate and methanol was added for 0.5 h at room temperature. Solvent was evaporated under reduced pressure. Product was dried under vacuum for 48 h. The resultant product was 0.2151 g (yield: 92.7%).

MS-ESI calcd for C₁₄O₂N₁H₁₆B(M⁺+H) 242.09; found 242.

MS-FAB calcd for C₁₇O₃N₁H₂₀B (M⁺+H) 298.09; found 298.

c. Synthesis of Diborono-Peptides in Solution

2-formylphenylboric acid (17.32 mg, 0.0488 mmol) was dissolved in 20 ml dried MeOH and then some molecular sieves (4 Å) were added. Then the peptide Ac-Ala-Ala-Lys-Ala-Ala-Lys-Ala (8.2 mg, 0.0122 mmol) was added. After 24 h at 40° C., NaBH₄ (0.92 mg, 0.0238 mmol) was added and the temperature was kept at 40° C. for 5 h. The desired product diborono-peptide was purified by preparative HPLC using a H₂O/Acetonitrile/0.05% TFA solvent system. (9.7 mg yield 84.5%). MALDI-MS (matrix DHB), calcd for C₅₇O₁₇N₉H₇₁B₂ (M+Na) 1198.85; found 1198.

4. Synthesis of Borono-Peptide Library

a. Library Synthesis of 12-mer Peptide Backbones

The synthetic procedure was described below.

(1). Weighed out 1 g (0.46 mmol/g, 110 μm) Nova TG aminomethyl PEG-PS resin and swelled it in HPLC grade DMF for 25 min.

(2). Dissolve 4.0 Equiv. of Fmoc-Ala-OH (573 mg, based on the loading of the resin) and the same Eqiuv. of HBTU (698 mg) in 10 ml 0.4 M N-methylmorpholine, then added the beads and tumble for 1 h.

(3). The beads was then washed with DMF and methanol (5×15 ml)

(4). After Kaiser test was negative, removed the protecting group of amine by the addition of 20% piperidine for 25 min. If Kaiser Test was positive, the resin beads were washed with DMF, methanol, and DCM respectively (5×15 ml) and the solvents were drained with aid of an aspirator.

(5). Distributed the beads into 5 reaction vessels with the ratio 6.1:1:1:1. Based on 4.0 Equiv, the following reagents were added into each reaction vessel respectively and tumble for 25-30 min.
Fmoc-Ala-OH (344 mg), HBTU (419 mg), and 0.4M N-methylmorpholine (8 ml)
Fmoc-Lys(Boc)-OH (86 mg), HBTU (70 mg), and 0.4M N-methylmorpholine (5 ml)
Fmoc-Orn(Boc)-OH (84 mg), HBTU (70 mg), and 0.4M N-methylmorpholine (5 ml)
Fmoc-DAB(Boc)-OH (81 mg), HBTU (70 mg), and 0.4M N-methylmorpholine (5 ml)
Fmoc-DPR(Boc)-OH (79 mg), HBTU (70 mg), and 0.4M N-methylmorpholine (5 ml)
   Kaiser Test was used to determine whether the coupling reaction was finished. The negative result (light brownish yellow) indicated the coupling was completed.

(6). The procedure was repeated from step (3) to (5) for 9 times.

(7) Finally, removed the N-terminal protecting group of the 11^{th} amino acid and capped it with Cbz-Ala-OH (274 mg) with HBTU (465 mg) and coupling for 1 h.

(8) The resin beads were washed with DMF, methanol, and DCM respectively (5×15 ml) If Kaiser test was negative, removed the side-chain protecting group using 95% TFA. The beads were washed by PBS and stored at 4° C.

b. Model Reaction 2

(1) Synthesis of 3-mer peptide for model reaction 2 ( Cbz-Ala-Lys-Ala-NH₂)

With the same synthetic method described above, the 3-mer peptide was synthesized. The amino acids and other raw materials were describes as follows: Fmoc-Ala-OH (125 mg); Fmoc-Lys(ivDde)-OH (230 mg); Cbz-Ala-OH (90 mg); HBTU (152 mg); HOBT (54 mg); Rink Am Resin (41 mg, 0.71 mmol/g). Finally, remove the side chain protecting group(iv Dde) using 2% hydrazine in DMF for 2 h.

(2) Synthesis of 3mer-borono-peptide for model reaction 2 in DMF/Methanol solvent system (9:1 v/v)

The boronic acid was incorporated into the peptide with reductive amination as described in 2.5.3 C. 2-formylphenylboric acid (9 mg, 0.125 mmol) was dissolved in 2.5 ml dried MeOH and then some molecular sieves (4 Å) were added to absorb water forming during the reaction 0.025 mmol 3-mer peptide beads were added with 22.5 ml dry DMF. After 24 h at 40° C., NaBH₄ (0.92 mg, 0.0238 mmol) was added and the temperature was kept at 40° C. for 5 h. This reaction was repeated to ensure the completion.

HPLC+MS-ESI calcd for C₂₇O₇N₅H₃₈B₁ (M⁺+H) 556.43; found 556. Spectrum showed a near complete coupling

(3) Synthesis of 3-mer borono-peptide using methanol solvent system

Except the solvent is methanol, all others were the same as the above. HPLC+MS-ESI for product calcd C₂₇O₇N₅H₃₈B₁ (M⁺+H) 556.43; found 556. HPLC showed an uncomplete coupling.

c. Synthesis of 12-Mer Borono-Peptide Library

2-formylphenylboronic acid (1.2 g, 8 mmol) was dissolved in the mixed solvent of dry methanol 2.5 ml and dry DMF 22.5 ml in a flask. About 0.35 g peptide library resin was added into the flask. With the same procedure described in the model reaction B, the coupling reaction was repeated twice for completion.

5. Synthesis of Conjugates of FITC-Ovalbumin, FITC-BSM, FITC-PSM and FITC-CEA

a. Conjugates of FITC-Ovalbumin, FITC-BSM, FITC-PSM

50 mg of ovalbumin was dissolved in 10 ml of 0.5 M carbonate buffer (pH=9.5) solution. Based on the ratio of 20 μg FITC/mg protein, 1 mg of FITC was dissolved in 1 ml of dry DMF. This FITC in DMF solution was then added into glycoprotein solution and incubated at 27° C. for 1 h. After that, the solution was dialyzed against 0.1 M PBS buffer (pH=7.3) in a cool room overnight. Then change fresh PBS buffer to dialyze for second time until aqueous solution was colorless. SDS-PAGE was used to confirm the formation of FITC-ovalbumin. Similarly, the conjugates of FITC-BSM and FITC-PSM were synthesized and purified. Analysis was completed using UV/Vis absorption to confirm the formation of FITC-BSM and FITC-PSM at wavelength 490 nm.

b. Synthesis of the Conjugate of FITC-Carcinoembryonic Antigen (CEA)

25 μg CEA was dissolved in 5 μl of 0.5 M carbonate buffer (pH=9.5) solution. Based on the ratio of 20 μg FITC/mg protein, needed FITC was 0.5 μg (converted to 1 μl of 0.5 mg/ml FITC in dry DMF). 1 μl of 0.5 mg/ml FITC/DMF solution was added into glycoprotein solution and incubated at 27° C. for 1 h. After that, FITC-CEA conjugate was purified through dialysis with Pierce Slide-A-Lyzer 3.5K dialysis cassettes against 0.1 M PBS buffer (pH=7.3) in a cool room overnight

6. Synthesis of Carboxyrhodamine Ester

20 mg crude carboxyrhodamine (purity 50%, 0.0206 mmol), 11.8 mg N-hydroxy-succinimide(NHS, 0.103 mmol) and 19.7 mg 1-ethyl-3-(3-dimethylaminopropyl)-carbodiimide hydrochloride (0.103 mmol) were dissolved in 1.5 ml dry DMF and the mixture was stirred for 5 h at 25 C. The product was used for labeling proteins without further purification.

7. Synthesis of the Conjugates of CR-PSM and CR-Ovalbumin

PSM (100 mg) was dissolved in 20 ml carbonate buffer (0.1 M, pH=8.5). 250 μl of rhodamine ester solution(synthesized in 2.5.6) was added and the mixture was incubated for 1 hour at 28° C. The resultant conjugate was purified through dialysis against carbonate buffer (0.1 M, pH=8.5) and PBS buffer (0.1 M, pH 7.3, 150 mM NaCl) in a cool room overnight.

In the same procedure and amount, the conjugate of Carboxyrhodamine-Ovalbumin was synthesized and purified.

8. Binding and Screening of PBL Library with Dye-Tagged Sugars

Resorufin-β-D-glucopyranoside and resorufin-β-D-galacopyranoside were dissolved in 1 ml DMSO respectively, then diluted with phosphate buffer (pH=7.2).The concentration of was 1.5 mM. Borono-peptide library beads were added into two Eppendorf tubes and then the sugar solution was added into the tubes respectively. The tubes were tumbled overnight. After that, the beads were washed with PBS buffer for a few times and then water was removed. The color change of the beads was observed under a microscope.

9. Binding and Screening of PBL Library with FITC/CR-Glycoproteins

FITC-glycoprotein or CR-glycoprotein solution was added into the borono-peptide beads and tumbled overnight at room temperature. The beads were then washed by PBS buffer (0.1M, pH=7.3 150 mM NaCl) (for CR-glycoproteins, beads were washed with methanol first and then washed with PBS buffer) and beads were observed under fluorescence microscope (Nikon, eclipse, E600) and pictures were taken.

### V. Use of PBL Library in Disease Diagnostics

Boronic acids have shown great utility in sensing simple sugars and complex glycoproteins. It is therefore reasonable that the differential display of phenylboronic acid (PBA) moieties on a peptide backbone would result in biocompatible, water soluble, cancer diagnostic, Peptide Borono Lectins (PBLs) (FIG. 2.24a), overcoming the limitations of previously described boronic acid based sensors. Herein is disclosed the design and synthesis of a PBL library; studies showing that selective and cross-reactive PBLs can be identified from this library; as well as experiments illustrating the utility of these compounds as disease diagnostics, specifically for cancer.

Novel Peptide Borono-Lectins (PBLs) have been synthesized and used to bind glycoproteins. Binding studies have shown that binding is glycoprotein and PBL dependent. The reversibility of the binding is demonstrated and used to determine selectivity patterns to identify selective and cross-reactive PBLs. The presented PBL sensors are highly significant and have the potential to revolutionize cancer diagnosis because their stability, biocompatibility, ease of synthesis, and ease of use will identify the aberrant glycosylation patterns correlated with tumorgenesis and metastasis.

The following investigation is intended to illustrate the use of the PBL library developed as a diagnostic tool for various diseases, including cancer. The illustration is not intended to limit the scope of the overall invention.

A ‘low’ diversity 12-mer PBL library was synthesized on aminomethyl PEG-PS resin (100 μm) using a biased split-and-pool combinatorial approach (FIG. 2.24b). Standard Fmoc synthetic schemes were followed with Dde protected side-chains. This resin was chosen because it is stable to acid hydrolysis, displays limited inherent fluorescence, and binds ligands comparably to that observed in solution. The general sequence of the library is: Cbz-A-(X)₁₀-A-resin; where X is either alanine (Ala), 2,3-diaminopropanoic acid (DPR), 2,4-diaminobutanoic acid (DAB), ornithine (Orn), or Lysine (Lys). These diamino acids contain 1, 2, 3, and 4 methylene units, respectively, spacing the side-chain amine from the peptide back-bone. Introduction of PBAs was accomplished by removing the Dde protecting groups with hydrazine, coupling the side-chain amine with excess 2-formylphenylboronic acid followed by reduction with NaBH₄. The theoretical diversity of the resulting PBL library (5¹⁰) is on the order of 10 million distinct peptide sequences containing a statistical average of 4 PBA moieties per peptide. Because 1 g of resin was used for library construction, the number of unique PBL sequences is approximately 2,000,000. Variation in the peptide sequence modifies the “horizontal” spacing between PBAs while varying the number of —CH₂— groups in the side-chains alters the “vertical” spacing. This creates distinct positional variation between PBAs within a sequence; thereby creating unique geometric constraints for binding.

To investigate the ability of the library to bind glycans, a series of fluorescently labeled glycoproteins (FITC) including ovalbumin (Oval), porcine stomach mucin (PSM) and bovine submaxillary mucin (BSM) was assayed. These glycoproteins were chosen because they are readily available and are enriched in high-mannose; hybrid and complex N-linked glycans as well as O-linked glycans—all motifs found in cancer-related targets. While labeling analytes is not useful for diagnostic applications, the use of tagged analytes is known to be an efficient and effective method for screening a resin-bound library.

To minimize non-specific binding, the resin was pre-incubated with 1% BSA. When binding glycoproteins to the library (about 10 min. to 12 hr—about 1 ng/mL to 1 mg/mL), 1% BSA and 10% glycerol were also included. The resin was washed extensively with phosphate buffered saline (PBS) after binding. All of these actions eliminate weak binders resulting in a 10% hit rate observed using a small portion of the library (˜100 beads).

Qualitative results from screening studies demonstrate that individual members of the PBL library can bind fluorescently labeled glycoproteins, FIG. 2.25. In contrast, unfunctionalized resin (no PBL attached) did not bind to any glycoprotein. Similarly, fluorescently labeled bovine serum albumin (BSA), which is not a glycoprotein, showed no appreciable binding to the PBL library. Likewise, BSM treated with hydrazine, to cleave all N-linked glycans, showed no binding to the PBL library (not shown); thereby demonstrating that binding was glycoprotein and PBL dependent. Glycoproteins were removed by washing with 2% NaOH (FIG. 2.25, 3^{rd} column) and then rebound to the same glycoprotein; thereby indicating that the PBL was not damaged during the wash protocol (FIG. 2.25, 4^{th} column).

To define selectivity, individual beads were placed in each well of a micro-titer plate and sequentially exposed to different targets (BSM, PSM, Oval). Beads were incubated with FITC-labeled glycoprotein, washed to remove unbound target, and binding imaged using a fluorescence microscope. Bound glycoprotein was washed away and the bead re-bound to the same target to show fidelity in binding. The glycoprotein was again washed away and the process was then repeated for the other glycoproteins. FIG. 2.26 shows images for four representative individual beads responding to different targets. Bead G2, showed selectivity for binding to only BSM. Likewise, bead E1 selectively bound Oval. However, bead H4 was partially cross-reactive; binding both BSM and PSM, and bead A6 was completely unselective, binding to all glycoproteins assayed.

Identification of selective PBLs is encouraging, yet cross-reactive PBLs are also useful for inclusion in array-based diagnostics. FIG. 2.27 schematically depicts the binding outcomes for each individual bead when bound to different targets. Colored circles represent a positive binding interaction between the PBL and the specific glycoprotein being screened. Grey circles were controls that showed no binding. White circles were PBLs that bound to no targets. These studies demonstrate that specific patterns (fingerprints) are generated for each glycoprotein. The individual color patterns shown in the top part of FIG. 2.27 indicate the array response to each glycoprotein while the composite image at the bottom of the figure schematically indicates selective and cross-reactive PBLs.

To demonstrate the utility of PBLs in binding cancer-specific targets, CEA was tagged with FITC and incubated with the PBL library at varying concentrations. FIG. 2.28 shows the microscope images for the library responding to 10 ng/mL and 10 pg/mL CEA. Higher sensitivity is achieved using longer integration times on the camera to image the beads, up to 25 ms to obtain the 10 pg/mL image. These results clearly indicate the utility for PBLs to act as diagnostics for the early detection of cancer.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

TABLE 2.2
Comparison of addition of glycerol

| Sample | Percentage of hits | | |
|---|---|---|---|
| PBL library bound 1000 μg/ml of FITC- | 24% | | 1 |
| ovalbumin, overnight | | | |
| PBL library bound 1000 μg/ml of FITC-ovalbumin + 5% (v/v) glycerol,overnight | 13% | 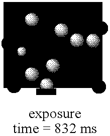 | 2 |
| PBL library bound 1000 μg/ml of FITC-ovalbumin + 10% (v/v) glycerol,overnight | 8% | 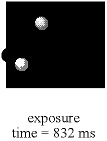 | 3 |
| PBL library was blocked with 1% BSA | 18% | | 4 |
| for 1 h, and then bound FITC-Oval | | | |
| overnight. | | | |

TABLE 2.3
Detection limits of a few fluorescently-labeled glycoproteins

| Fluorescence reagent | FITC | | | CR | |
|---|---|---|---|---|---|
| Exposure time | 252 ms | | | 62.4 ms | |
| Glycoprotein | Oval | BSM | CEA | Oval | PSM |
| Concentration limit | 10 | 10 | 0.1 | 1 | 1 |
| (μg/ml) | | | | | |

TABLE 2.4
Binding sequence of PBL arrays

| | Plate 1 | Plate 2 | Plate 3 |
|---|---|---|---|
| Started from | Oval | BSM | PSM |

Washing with 1M NaOH overnight followed washing with PBS

| | | | |
|---|---|---|---|
| Rebound | Oval | BSM | PSM |

Washing with 1M NaOH overnight followed washing with PBS

| | | | |
|---|---|---|---|
| Bound | BSM | PSM | Oval |

Washing with 1M NaOH overnight followed washing with PBS

| | | | |
|---|---|---|---|
| Bound | PSM | Oval | BSM |

Washing with 1M NaOH overnight followed washing with PBS
Take fluorescence images

What is claimed:

1. A method of detecting the presence of a carbohydrate in an aqueous sample, the method comprising
applying a sample to a biosensor, wherein the biosensor comprises a boronic acid moiety bonded to a protein scaffold, wherein the boronic acid moiety bonded to the protein scaffold is configured to bond to a particular carbohydrate of interest; and
determining whether the particular carbohydrate of interest bonded to the boronic acid moiety.

2. A method as in claim 1, wherein the boronic acid moiety bonded to the protein scaffold comprises a boronic acid functional group bonded to an amino acid.

3. A method as in claim 2, wherein the amino acid having the bonded boronic acid functional group is present within a backbone of the protein scaffold.

4. A method as in claim 2, wherein the amino acid having the bonded boronic acid functional group is present on a side chain extending off of the protein scaffold.

5. A method as in claim 1, wherein biosensor comprises a plurality of boronic acid moieties bonded to the protein scaffold.

6. A method as in claim 5, wherein the plurality of boronic acid moieties are positioned on the protein scaffold such that the boronic acid moieties bond to the particular carbohydrate of interest.

7. A method as in claim 1, wherein the particular carbohydrate of interest is part of a glycoprotein.

8. A method as in claim 7, wherein the glycoprotein is a cancer-associated glycoprotein.

9. A method as in claim 7, wherein the presence of the particular carbohydrate of interest in a sample provided from the human is indicative that the human is suffering from a particular decease.

10. A biosensor for determining the presence of an analyte in a sample, the biosensor comprising
a protein scaffold, wherein the protein scaffold comprises a plurality of amino acids; and
a boronic acid moiety bonded to one of the amino acids of the protein scaffold.

11. A biosensor as in claim 10 further comprising a plurality of boronic acid moieties, wherein the boronic acid moieties are positioned on the protein scaffold such that the biosensor bonds to a targeted analyte.

12. A biosensor as in claim 10, wherein the boronic acid moiety is bonded to an amino acid located on a backbone of the protein scaffold.

13. A biosensor as in claim 10, wherein the boronic acid moiety is bonded to an amino acid located on a side chain extending from the protein scaffold.

14. A biosensor as in claim 10, wherein the boronic acid moiety bonded to the protein scaffold comprises a boronic acid functional group bonded to an amino acid.

15. A biosensor as in claim 10, wherein the targeted analyte is a carbohydrate.

16. A biosensor as in claim 15, wherein the carbohydrate is part of a glycoprotein.

17. A biosensor as in claim 16, wherein the glycoprotein is a cancer-associated glycoprotein.

18. A biosensor as in claim 10, wherein the protein scaffold comprises less than about 30 amino acids.
